SEQUENCE LISTING (75 sequences)

SEQ ID NO: 1 - protein, 5 aa, synthetic construct
DYNMA

SEQ ID NO: 2 - protein, 17 aa, synthetic construct
TIIYDGGSTYYRHSVKG

SEQ ID NO: 3 - protein, 12 aa, synthetic construct
QTYFGSRDYFDY

SEQ ID NO: 4 - protein, 11 aa, synthetic construct
LTSEDIYNNLA

SEQ ID NO: 5 - protein, 7 aa, synthetic construct
YASNFQD

SEQ ID NO: 6 - protein, 8 aa, synthetic construct
LQDSEYPP

SEQ ID NO: 7 - protein, 121 aa, synthetic construct
EVQLVESGGGLVQPGRSLKLSCAASGFTFSDYNMAWVRQAPKKGLEWVATIIYDGGSTYYRHSVKGRFTISRDNAKSTHSLQMDSLRSEDTATYYCARQTYFGSRDYFDYWGQGVMVTVSS

SEQ ID NO: 8 - protein, 108 aa, synthetic construct
DIQMTQSPTSLSASLGETVSIECLTSEDIYNNLAWYQQKPGKSPQLLISYASNFQDGVPSRFSGSGSGTQYSLKINSLESEDAATYFCLQDSEYPPTFGGGTKLELKR

SEQ ID NO: 9 - protein, 451 aa, synthetic construct
EVQLVESGGGLVQPGRSLKLSCAASGFTFSDYNMAWVRQAPKKGLEWVATIIYDGGSTYYRHSVKGRFTISRDNAKSTHSLQMDSLRSEDTATYYCARQTYFGSRDYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPTSLSASLGETVSIECLTSEDIYNNLAWYQQKPGKSPQLLISYASNFQDGVPSRFSGSGSGTQYSLKINSLESEDAATYFCLQDSEYPPTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
SYGMH

SEQ ID NO: 12 - protein, 17 aa, synthetic construct
VIWYDGSNKYYADSVKG

SEQ ID NO: 13 - protein, 9 aa, synthetic construct
DSSPYGMDV

SEQ ID NO: 14 - protein, 11 aa, synthetic construct
RASQGISSWLA

SEQ ID NO: 15 - protein, 7 aa, synthetic construct
GASSLQS

SEQ ID NO: 16 - protein, 9 aa, synthetic construct
QQSDSFPRT

SEQ ID NO: 17 - protein, 118 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDSSPYGMDVWGQGTTVTVSS

SEQ ID NO: 18 - protein, 108 aa, synthetic construct
DIQMTQSPSSVSASVGDRVTITCRASQGISSWLAWYQQKPGKAPKLLIYGASSLQSGVPSRFSASGSGTDFTLTISSLQPEDFATYYCQQSDSFPRTFGQGTKVEIKR

SEQ ID NO: 19 - protein, 448 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDSSPYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPCVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
DIQMTQSPSSVSASVGDRVTITCRASQGISSWLAWYQQKPGKAPKLLIYGASSLQSGVPSRFSASGSGTDFTLTISSLQPEDFATYYCQQSDSFPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
DTYIH

SEQ ID NO: 22 - protein, 17 aa, synthetic construct
RIDPANGNTKYDPKFQD

SEQ ID NO: 23 - protein, 11 aa, synthetic construct
NYGSNYDPMDY

SEQ ID NO: 24 - protein, 11 aa, synthetic construct
RASQNIDTNIH

SEQ ID NO: 25 - protein, 7 aa, synthetic construct
YASESIS

SEQ ID NO: 26 - protein, 9 aa, synthetic construct
QQSDTWPWT

SEQ ID NO: 27 - protein, 5 aa, synthetic construct
DYSVH

SEQ ID NO: 28 - protein, 16 aa, synthetic construct
IMWTGGSTAYNSALKS

SEQ ID NO: 29 - protein, 8 aa, synthetic construct
DGDYGPDY

SEQ ID NO: 30 - protein, 11 aa, synthetic construct
QASQDIGNWLS

SEQ ID NO: 31 - protein, 7 aa, synthetic construct
GATSLAD

SEQ ID NO: 32 - protein, 10 aa, synthetic construct
LQAYSAPPWT

SEQ ID NO: 33 - protein, 120 aa, synthetic construct
ELQLQQSGAELVRPGASVKLSCTTSGFNVKDTYIHWVRQRPEQGLEWIGRIDPANGNTKYDPKFQDRATITTDTSSITAYLQLSSLTSEDTAVYYCARNYGSNYDPMDYWGQGTSLTVSS

SEQ ID NO: 34 - protein, 108 aa, synthetic construct
DILLTQSPAILSVSPGERVSFSCRASQNIDTNIHWYQRRTNDSPRLLIKYASESISGIPSRFSGSGSGTDFTLSINSVESEDIADYYCQQSDTWPWTFGGGTKLEIKR

SEQ ID NO: 35 - protein, 116 aa, synthetic construct
EVQLKESGPGLVQPSQTLSLTCTVSGFSLTDYSVHWVRQSPGKGLEWMGIMWTGGSTAYNSALKSRLSISRDTSKSQVFLKMNSLQTEDTAIYYCTRDGDYGPDYWGQGVMVTVSS

SEQ ID NO: 36 - protein, 109 aa, synthetic construct
DIQMTQSPASLSASLEEIVTITCQASQDIGNWLSWYQQKPGKSPQLLIYGATSLADGVPSRFSGSRSGTQYSLKISRLQVEDIGIYYCLQAYSAPPWTFGGGTKLELKR

SEQ ID NO: 37 - protein, 495 aa, synthetic construct
ELQLQQSGAELVRPGASVKLSCTTSGFNVKDTYIHWVRQRPEQGLEWIGRIDPANGNTKYDPKFQDRATITTDTSSITAYLQLSSLTSEDTAVYYCARNYGSNYDPMDYWGQGTSLTVSSGGGGSGGGGSGGGGSDILLTQSPAILSVSPGERVSFSCRASQNIDTNIHWYQRRTNDSPRLLIKYASESISGIPSRFSGSGSGTDFTLSINSVESEDIADYYCQQSDTWPWTFGGGTKLEIKRSGGGGSEVQLKESGPGLVQPSQTLSLTCTVSGFSLTDYSVHWVRQSPGKGLEWMGIMWTGGSTAYNSALKSRLSISRDTSKSQVFLKMNSLQTEDTAIYYCTRDGDYGPDYWGQGVMVTVSSGGGGSGGGGSGGGGSDIQMTQSPASLSASLEEIVTITCQASQDIGNWLSWYQQKPGKSPQLLIYGATSLADGVPSRFSGSRSGTQYSLKISRLQVEDIGIYYCLQAYSAPPWTFGGGTKLELKRHHHHHH

SEQ ID NO: 38 - protein, 5 aa, synthetic construct
SYGMH

SEQ ID NO: 39 - protein, 17 aa, synthetic construct
VIWYDGSNKYYADSVKG

SEQ ID NO: 40 - protein, 9 aa, synthetic construct
DSSPYGMDV

SEQ ID NO: 41 - protein, 11 aa, synthetic construct
RASQGISSWLA

SEQ ID NO: 42 - protein, 7 aa, synthetic construct
GASSLQS

SEQ ID NO: 43 - protein, 9 aa, synthetic construct
QQSDSFPRT

SEQ ID NO: 44 - protein, 118 aa, synthetic construct; identical to SEQ ID NO: 17

SEQ ID NO: 45 - protein, 108 aa, synthetic construct; identical to SEQ ID NO: 18

SEQ ID NO: 46 - protein, 496 aa, synthetic construct
ELQLQQSGAELVRPGASVKLSCTTSGFNVKDTYIHWVRQRPEQGLEWIGRIDPANGNTKYDPKFQDRATITTDTSSITAYLQLSSLTSEDTAVYYCARNYGSNYDPMDYWGQGTSLTVSSGGGGSGGGGSGGGGSDILLTQSPAILSVSPGERVSFSCRASQNIDTNIHWYQRRTNDSPRLLIKYASESISGIPSRFSGSGSGTDFTLSINSVESEDIADYYCQQSDTWPWTFGGGTKLEIKRSGGGGSQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDSSPYGMDVWGQGTTVTVSSGGGGSGGGGSGGGGSDIQMTQSPSSVSASVGDRVTITCRASQGISSWLAWYQQKPGKAPKLLIYGASSLQSGVPSRFSASGSGTDFTLTISSLQPEDFATYYCQQSDSFPRTFGQGTKVEIKRHHHHHH

SEQ ID NO: 47 - protein, 22 aa, synthetic construct
MDMRVPAQLLGLLLLWLRGARC

SEQ ID NO: 48 - protein, 66 aa, synthetic construct
ATGGACATGAGAGTGCCTGCACAGCTGCTGGGCCTGCTGCTGCTGTGGCTGAGAGGCGCCAGATGC

SEQ ID NO: 49 - protein, 19 aa, synthetic construct
MAWALLLLTLLTQGTGSWA

SEQ ID NO: 50 - protein, 57 aa, synthetic construct
ATGGCCTGGGCTCTGCTGCTCCTCACCCTCCTCACTCAGGGCACAGGGTCCTGGGCC

SEQ ID NO: 51 - protein, 444 aa, synthetic construct
ELQLQQSGAELVRPGASVKLSCTTSGFNVKDTYIHWVRQRPEQGLEWIGRIDPANGNTKYDPKFQDRATITTDTSSITAYLQLSSLTSEDTAVYYCARNYGSNYDPMDYWGQGTSLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 52 - protein, 214 aa, synthetic construct
DILLTQSPAILSVSPGERVSFSCRASQNIDTNIHWYQRRTNDSPRLLIKYASESISGIPSRFSGSGSGTDFTLSINSVESEDIADYYCQQSDTWPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 53 - protein, 710 aa, synthetic construct
EQLEESGGDLVKPGASLTLTCTASGFSFTSDYYMCWVRQAPGKGLEWIACIGAGDIHTTYYANWAKGRFTISKTSSTTVTLQMTTLTAADTATYFCARDTYNIGGYTGDFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 217 aa, synthetic construct
ALVMTQPPASVSAAVGGTVTINCQASESISTWLAWYQQKPGQPPKLLIYYASTLASGVPSRFKGSGSGTQFTLTISGVECDDAATYYCAGHKSYSSDDFAFGGGTEVVVKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 710 aa, synthetic construct
EQLEESGGDLVKPGASLTLTCTASGFSFTSDYYMCWVRQAPGKGLEWIACHGAGDIHTTYYANWAKGRFTISKTSSTTVTLQMTTLTAADTATYFCARDTYNIGGYTGDFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 217 aa, synthetic construct; identical to SEQ ID NO: 54

SEQ ID NO: 57 - protein, 710 aa, synthetic construct
EQLEESGGDLVKPGASLTLTCTASGFSFTSDYYHCWVRQAPGKGLEWIACIGAGDIHTTYYANWAKGRFTISKTSSTTVTLQMTTLTAADTATYFCARDTYNIGGYTGDFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 217 aa, synthetic construct; identical to SEQ ID NO: 54

SEQ ID NO: 59 - protein, 6 aa, synthetic construct
SDYYMC

SEQ ID NO: 60 - protein, 18 aa, synthetic construct
CIGAGDIHTTYYANWAKG

SEQ ID NO: 61 - protein, 14 aa, synthetic construct
DTYNIGGYTGDFDL

SEQ ID NO: 62 - protein, 11 aa, synthetic construct
QASESISTWLA

SEQ ID NO: 63 - protein, 7 aa, synthetic construct
YASTLAS

SEQ ID NO: 64 - protein, 12 aa, synthetic construct
AGHKSYSSDDFA

SEQ ID NO: 65 - protein, 6 aa, synthetic construct
SDYYMC

SEQ ID NO: 66 - protein, 18 aa, synthetic construct
CHGAGDIHTTYYANWAKG

SEQ ID NO: 67 - protein, 14 aa, synthetic construct
DTYNIGGYTGDFDL

SEQ ID NO: 68 - protein, 6 aa, synthetic construct
SDYYHC

SEQ ID NO: 69 - protein, 18 aa, synthetic construct
CIGAGDIHTTYYANWAKG

SEQ ID NO: 70 - protein, 14 aa, synthetic construct
DTYNIGGYTGDFDL

SEQ ID NO: 71 - protein, 123 aa, synthetic construct
EQLEESGGDLVKPGASLTLTCTASGFSFTSDYYMCWVRQAPGKGLEWIACIGAGDIHTTYYANWAKGRFTISKTSSTTVTLQMTTLTAADTATYFCARDTYNIGGYTGDFDLWGPGTLVTVSS

SEQ ID NO: 72 - protein, 111 aa, synthetic construct
ALVMTQPPASVSAAVGGTVTINCQASESISTWLAWYQQKPGQPPKLLIYYASTLASGVPSRFKGSGSGTQFTLTISGVECDDAATYYCAGHKSYSSDDFAFGGGTEVVVKG

SEQ ID NO: 73 - protein, 123 aa, synthetic construct
EQLEESGGDLVKPGASLTLTCTASGFSFTSDYYMCWVRQAPGKGLEWIACHGAGDIHTTYYANWAKGRFTISKTSSTTVTLQMTTLTAADTATYFCARDTYNIGGYTGDFDLWGPGTLVTVSS

SEQ ID NO: 74 - protein, 123 aa, synthetic construct
EQLEESGGDLVKPGASLTLTCTASGFSFTSDYYHCWVRQAPGKGLEWIACIGAGDIHTTYYANWAKGRFTISKTSSTTVTLQMTTLTAADTATYFCARDTYNIGGYTGDFDLWGPGTLVTVSS

SEQ ID NO: 75 - protein, 502 aa, synthetic construct
EQLEESGGDLVKPGASLTLTCTASGFSFTSDYYMCWVRQAPGKGLEWIACIGAGDIHTTYYANWAKGRFTISKTSSTTVTLQMTTLTAADTATYFCARDTYNIGGYTGDFDLWGPGTLVTVSSSGGGGSGGGGSGGGGSALVMTQPPASVSAAVGGTVTINCQASESISTWLAWYQQKPGQPPKLLIYYASTLASGVPSRFKGSGSGTQFTLTISGVECDDAATYYCAGHKSYSSDDFAFGGGTEVVVKGSGGGGSEVQLKESGPGLVQPSQTLSLTCTVSGFSLTDYSVHWVRQSPGKGLEWMGIMWTGGSTAYNSALKSRLSISRDTSKSQVFLKMNSLQTEDTAIYYCTRDGDYGPDYWGQGVMVTVSSGGGGSGGGGSGGGGSDIQMTQSPASLSASLEEIVTITCQASQDIGNWLSWYQQKPGKSPQLLIYGATSLADGVPSRFSGSRSGTQYSLKISRLQVEDIGIYYCLQAYSAPPWTFGGGTKLELKRHHHHHH

(19) United States
(12) Patent Application Publication
(10) Pub. No.: US 2026/0042865 A1
(43) Pub. Date: Feb. 12, 2026

(54) MULTISPECIFIC MOLECULES FOR CLEARANCE OF IMMUNOGLOBULINS IN THE TREATMENT OF AUTOANTIBODY-INDUCED DISEASES

(71) Applicant: AMGEN INC., Thousand Oaks, CA (US)

(72) Inventors: Venkata Siva Charan DEVANABOYINA, Fremont, CA (US); Peng LI, San Francisco, CA (US)

(73) Assignee: AMGEN INC., Thousand Oaks, CA (US)

(21) Appl. No.: 19/124,182

(22) PCT Filed: Oct. 25, 2023
(86) PCT No.: PCT/US2023/077772

§ 371 Date: Apr. 24, 2025

Related U.S. Application Data
(60) Provisional application No. 63/419,549, filed on Oct. 26, 2022.

(51) Int. Cl. C07K 16/42; A61K 39/00; A61P 37/06; C07K 16/28

(52) U.S. Cl. CPC C07K 16/4241; A61P 37/06; C07K 16/2851; A61K 2039/505; C07K 2317/31; C07K 2317/622; C07K 2317/92; C07K 2317/94

(57) ABSTRACT

The present invention is directed to multispecific molecules that bind immunoglobulin and a recycling target. Binding immunoglobulin and a recycling target results in degradation of immunoglobulin and in certain embodiments recycling of the multispecific molecule. The multispecific molecules of the present invention are thought to be useful in the treatment of autoantibody-induced diseases.

### CROSS-REFERENCE TO RELATED APPLICATIONS

This application claims the benefit of U.S. Provisional Application No. 63/419,549, filed Oct. 26, 2022, which is hereby incorporated by reference in its entirety.

### DESCRIPTION OF THE TEXT FILE SUBMITTED ELECTRONICALLY

The present application contains a Sequence Listing, which has been submitted electronically in XML format and is hereby incorporated by reference in its entirety. The computer readable format copy of the Sequence Listing, which was created on Oct. 25, 2023, is named 10179-WO01-SEC_ST26 and is 72.1 kilobytes in size.

### FIELD OF THE INVENTION

The present invention relates to the field of autoantibody-induced diseases. The present invention relates to multispecific molecules, such as bispecific scFv molecules that bind immunoglobulin and a recycling target. Multispecific molecules of the present invention are useful in the treatment of autoantibody-induced diseases.

### BACKGROUND OF THE INVENTION

More than 2.5% of the world population is affected by autoantibody driven immune diseases (Lenti et al., Autoimmunity Rev. September 2022; 21(9): 103143). Due to immune system failures, antibodies generated against self-antigens, known as autoantibodies, induce pathogenic effects by various mechanisms such as blockade of function, modifying antigen trafficking mechanism, degradation of antigen, and activation of complement at the site of binding. Autoantibodies play a central role in disease pathology and therefore considerable efforts have been made to inhibit the production of antibodies or to deplete them from the circulation.

Predominantly, in various autoimmune disorders, autoantibodies are of IgG sub-class, which bind to FcRn (neonatal Fc receptor) in a pH-dependent manner through their constant region (Fc). This pH-dependent interaction of FcRn:IgG enables binding of internalized IgGs to FcRn in early endosomes and trafficking them back to the cell surface. Salvaging from lysosomal degradation results in long serum half-life for IgGs. Blocking the FcRn:Fc interaction therefore increases shunting of IgGs to lysosomal compartments, thereby enhancing the degradation of IgGs.

A wide variety of FcRn inhibitors are being developed and are either approved or in late-stage clinical trials to treat autoantibody mediated disorders. It has been demonstrated that blocking the FcRn:Fc interactions decreases total IgG levels both in the clinic and in pre-clinical animals. FcRn inhibitors have demonstrated clinical efficacy by depleting IgGs in circulation, but depletion of IgGs is not instantaneous after administering the inhibitors. The effect of FcRn inhibitors relies on endocytosis of IgGs, which is a rate-limiting process. It has been observed in the clinic that approximately three to four weeks are required to achieve depletion of about 50% of total and antigen specific IgGs post administration of FcRn inhibitors. Additionally, FcRn inhibitors must be administered with high frequency and/or at high doses to achieve this efficacy.

To overcome this delay and to induce rapid depletion of serum IgGs, multispecific molecules of the present invention were engineered. The multispecific molecules bind immunoglobulin and a recycling target and result in rapid lysosomal degradation of immunoglobulin. Multispecific molecules may be pH/Ca2+-dependent or independent. In addition to rapid depletion of immunoglobulins, the multispecific molecules of the present invention are expected to be advantageous in clearing immunoglobulin such as IgG by negating the formation of IgG-complexes, thereby resulting in reduced off-target effects.

### SUMMARY OF THE INVENTION

In one aspect, the present invention is directed to a multispecific molecule comprising a first binding domain and a second binding domain, wherein the first binding domain specifically binds immunoglobulin, and the second binding domain specifically binds a recycling target. In certain embodiments, the first binding domain is an scFv, Fv, scFab, Fab′, or Fab, and the second binding domain is an scFv, Fv, scFab, Fab′, or Fab. In particular embodiments, the first binding domain and/or second binding domain is an scFv. In certain such embodiments, the first binding domain and the second binding domain are each an scFv. In particular embodiments, the first binding domain and/or second binding domain is an Fv. In certain such embodiments, the first binding domain and the second binding domain are each an Fv. In certain embodiments, the first binding domain and/or second binding domain is an scFab. In certain such embodiments, the first binding domain and the second binding domain are each an scFab. In certain embodiments, the first binding domain and/or second binding domain is a Fab. In certain such embodiments, the first binding domain and the second binding domain are each a Fab. In certain embodiments, the first binding domain is an scFv and the second binding domain is a Fab. In certain embodiments, the first binding domain is an scFv and the second binding domain is a scFab. In certain embodiments, the first binding domain is a Fab and the second binding domain is an scFv. In certain embodiments, the first binding domain is a scFab and the second binding domain is an scFv. In certain embodiments, the first binding domain is a Fab and the second binding domain is an scFab. In certain embodiments, the first binding domain is a scFab and the second binding domain is a Fab. In certain embodiments, the first binding domain is an scFv and the second binding domain is an Fv. In certain embodiments, the first binding domain is an Fv and the second binding domain is an scFv. In certain embodiments, the first binding domain is an scFab and the second binding domain is an Fv. In certain embodiments, the first binding domain is an Fv and the second binding domain is an scFab. In certain embodiments, the first binding domain is an Fv and the second binding domain is a Fab. In certain embodiments, the first binding domain is a Fab and the second binding domain is an Fv. In certain embodiments, the first binding domain is an scFv and the second binding domain is a Fab′. In certain embodiments, the first binding domain is a Fab′ and the second binding domain is an scFv. In certain embodiments, the first binding domain is a Fab′ and the second binding domain is an Fv. In certain embodiments, the first binding domain is an Fv and the second binding domain is a Fab′. In certain embodiments, the first binding domain is a Fab′ and the second binding domain is an scFab. In certain embodiments, the first binding domain is an scFab and the second binding domain is a Fab′. In certain embodiments, the first binding domain and the second binding domain are each a Fab′. In certain embodiments, the first binding domain is a Fab and the second binding domain is a Fab′. In certain embodiments, the first binding domain is a Fab′ and the second binding domain is a Fab.

In certain embodiments, a single chain polypeptide comprises the first binding domain and the second binding domain.

In certain embodiments, the first binding domain and second binding domain are connected via a linker. In certain embodiments, the linker is a polypeptide linker. In certain embodiments, the linker is a SG₄S linker. In particular embodiments, the SG₄S linker comprises one SG₄S connecting the two binding domains. In other embodiments, the SG₄S linker comprises two SG₄S repeats. In certain embodiments, the SG₄S linker comprises three SG₄S repeats. In other embodiments, the SG₄S linker comprises four SG₄S repeats. In other embodiments, the SG₄S linker comprises five SG₄S repeats. In other embodiments, the SG₄S linker comprises six SG₄S repeats. In other embodiments, the SG₄S linker comprises seven or more SG₄S repeats. In certain embodiments, the linker comprises a sequence selected from the group consisting of (Gly₃Ser)₃ (SEQ ID NO: 76), (Gly₄Ser)₃ (SEQ ID NO: 77), (Gly₃Ser)₄ (SEQ ID NO: 78), (Gly₄Ser)₄ (SEQ ID NO: 79), (Gly₃Ser)₅ (SEQ ID NO: 80), (Gly₄Ser)₅ (SEQ ID NO: 81), (Gly₃Ser)₆ (SEQ ID NO: 82), (Gly₄Ser)₆ (SEQ ID NO: 83), GSADDAKKDAAKKDAAKKDDAKKDDAGS (SEQ ID NO: 84), GSADDAKKDAAKKDAAKKDDAKKDDAKKDAGS (SEQ ID NO: 85), (Gly₃Gln)₂ (SEQ ID NO: 86), (Gly₄Gln)₂ (SEQ ID NO: 87), (Gly₃Gln)₃ (SEQ ID NO: 88), (Gly₄Gln)₃ (SEQ ID NO: 89), (Gly₃Gln)₄ (SEQ ID NO: 90), (Gly₄Gln)₄ (SEQ ID NO: 91), (Gly₃Gln)₅ (SEQ ID NO: 92), (Gly₄Gln)₅ (SEQ ID NO: 93), (Gly₃Gln)₆ (SEQ ID NO: 94), (Gly₄Gln)₆ (SEQ ID NO: 95), (Gly₃Ser)₂ (SEQ ID NO: 96), and (Gly₄Ser)₂ (SEQ ID NO: 97).

In certain embodiments, the first binding domain specifically binds immunoglobulin. In certain embodiments, the first binding domain specifically binds a recycling target. In certain embodiments, the second binding domain specifically binds immunoglobulin. In certain embodiments, the second binding domain specifically binds a recycling target.

In certain embodiments, the multispecific molecules of the present invention specifically binds immunoglobulin, wherein the bound immunoglobulin is IgG, IgA, IgE, IgD, or IgM. In certain embodiments, the bound immunoglobulin is IgG. In certain embodiments, the bound immunoglobulin is IgA. In certain embodiments, the bound immunoglobulin is IgE. In certain embodiments, the bound immunoglobulin is IgD. In certain embodiments, the bound immunoglobulin is IgM. In certain embodiments, the bound immunoglobulin is expressed on a B cell. In certain embodiments, the bound immunoglobulin is expressed on a plasma cell. In certain embodiments, the bound immunoglobulin is circulating in blood.

In certain embodiments, the multispecific molecules of the present invention specifically binds a recycling target, wherein the recycling target is ASGR1. In certain embodiments, the recycling target is transferrin receptor. In certain embodiments, the recycling target is mannose 6 phosphate receptor.

In certain embodiments, the multispecific molecule of the present invention depletes at least 50%, 55%, 60%, 65%, 70%, 75%, 80%, 85%, 90%, 95%, 98%, 99%, or 100% of bound immunoglobulin in vivo. In particular embodiments, the multispecific molecule depletes about 50% to about 70% of immunoglobulin. In particular embodiments, the multispecific molecule depletes at least 50% of immunoglobulin. In particular embodiments, the multispecific molecule depletes at least 55% of immunoglobulin. In particular embodiments, the multispecific molecule depletes at least 60% of immunoglobulin. In particular embodiments, the multispecific molecule depletes at least 65% of immunoglobulin. In particular embodiments, the multispecific molecule depletes at least 70% of immunoglobulin. In particular embodiments, the multispecific molecule depletes at least 75% of immunoglobulin. In particular embodiments, the multispecific molecule depletes at least 80% of immunoglobulin. In particular embodiments, the multispecific molecule depletes at least 85% of immunoglobulin. In particular embodiments, the multispecific 4a molecule depletes at least 90% of immunoglobulin. In particular embodiments, the multispecific molecule depletes at least 95% of immunoglobulin. In particular embodiments, the multispecific molecule depletes at least 98% of immunoglobulin. In particular embodiments, the multispecific molecule depletes at least 99% of immunoglobulin. In particular embodiments, the multispecific molecule depletes 100% of immunoglobulin. In certain embodiments, the immunoglobulin is depleted in mice. In certain embodiments, the immunoglobulin is depleted in non-human primates. In certain embodiments, the immunoglobulin is depleted in human patients. In certain embodiments, the immunoglobulin is IgG. In certain embodiments, the immunoglobulin is IgA. In certain embodiments, the immunoglobulin is IgE. In certain embodiments, the immunoglobulin is IgD. In certain embodiments, the immunoglobulin is IgM. In certain embodiments, the immunoglobulin is depleted in less than 96 hours of administration. In certain embodiments, the immunoglobulin is depleted in less than 72 hours of administration. In certain embodiments, the immunoglobulin is depleted within 3 hours to 96 hours of administration. In certain embodiments, the immunoglobulin is depleted within 3 hours to 72 hours of administration.

In certain embodiments, the multispecific molecule of the present invention binds in a catabolic manner to a recycling target and in a non-catabolic manner to Ig.

In certain embodiments, the multispecific molecule of the present invention binds in a catabolic manner to a recycling target and in a catabolic manner to Ig.

In certain embodiments, the multispecific molecule of the present invention binds in a non-catabolic manner to a recycling target and in a catabolic manner to Ig.

In certain embodiments, the multispecific molecule of the present invention remains bound to the immunoglobulin and the recycling target.

In certain embodiments, the multispecific molecule of the present invention dissociates from the immunoglobulin and the recycling target in an endosome of a cell that expresses the recycling target.

In certain embodiments, the multispecific molecule of the present invention dissociates from the recycling target in an endosome of a cell that expresses the recycling target.

In certain embodiments, the multispecific molecule of the present invention dissociates from the immunoglobulin in an endosome of a cell that expresses the recycling target. In certain such embodiments, the multispecific molecule remains bound to the recycling target in the endosome and is recycled to the cell surface of the cell that expresses the recycling target.

In another aspect, the present invention is directed to an antibody that specifically binds ASGR1, comprising a heavy chain (HC) and a light chain (LC), wherein the HC comprises a heavy chain variable region (HCVR) and the LC comprises a light chain variable region (LCVR), wherein the LCVR comprises LCDR1, LCDR2, and LCDR3, wherein HCDR1 comprises an amino acid sequence given by SEQ ID NO: 1, HCDR2 comprises an amino acid sequence given by SEQ ID NO: 2, HCDR3 comprises an amino acid sequence given by SEQ ID NO: 3, LCDR1 comprises an amino acid sequence given by SEQ ID NO: 4, LCDR2 comprises an amino acid sequence given by SEQ ID NO: 5, and LCDR3 comprises an amino acid sequence given by SEQ ID NO: 6. In certain embodiments, the HCVR comprises an amino acid sequence given by SEQ ID NO: 7. In certain embodiments, the LCVR comprises an amino acid sequence given by SEQ ID NO: 8. In certain embodiments, the HC comprises an amino acid sequence given by SEQ ID NO: 9. In certain embodiments, the LC comprises an amino acid sequence given by SEQ ID NO: 10. In particular embodiments, the antibody of the present invention is non-catabolic.

In certain embodiments, the multispecific molecule of the present invention comprises a binding domain that specifically binds ASGR1, and wherein said binding domain comprises a HCVR and LCVR, wherein the HCVR comprises HCDR1, HCDR2, and HCDR3, and wherein the LCVR comprises LCDR1, LCDR2, and LCDR3, wherein HCDR1 comprises an amino acid sequence given by SEQ ID NO: 1, HCDR2 comprises an amino acid sequence given by SEQ ID NO: 2, HCDR3 comprises an amino acid sequence given by SEQ ID NO: 3, LCDR1 comprises an amino acid sequence given by SEQ ID NO: 4, LCDR2 comprises an amino acid sequence given by SEQ ID NO: 5, and LCDR3 comprises an amino acid sequence given by SEQ ID NO: 6. In certain embodiments, the HCVR comprises an amino acid sequence given by SEQ ID NO: 7. In certain embodiments, the LCVR comprises an amino acid sequence given by SEQ ID NO: 8. In particular embodiments, the multispecific molecule of the present invention is non-catabolic.

The present invention provides an antibody that specifically binds ASGR1, comprising a heavy chain (HC) and a light chain (LC), wherein the HC comprises a heavy chain variable region (HCVR) and the LC comprises a light chain variable region (LCVR), wherein the LCVR comprises LCDR1, LCDR2, and LCDR3, wherein HCDR1 comprises an amino acid sequence given by SEQ ID NO: 11, HCDR2 comprises an amino acid sequence given by SEQ ID NO: 12, HCDR3 comprises an amino acid sequence given by SEQ ID NO: 13, LCDR1 comprises an amino acid sequence given by SEQ ID NO: 14, LCDR2 comprises an amino acid sequence given by SEQ ID NO: 15, and LCDR3 comprises an amino acid sequence given by SEQ ID NO: 16. In certain embodiments, the HCVR comprises an amino acid sequence given by SEQ ID NO: 17. In certain embodiments, the LCVR comprises an amino acid sequence given by SEQ ID NO: 18. In certain embodiments, the HC comprises an amino acid sequence given by SEQ ID NO: 19. In certain embodiments, the LC comprises an amino acid sequence given by SEQ ID NO: 20. In particular embodiments, the antibody of the present invention is catabolic.

In certain embodiments, the multispecific molecule of the present invention comprises a binding domain that specifically binds ASGR1, and wherein said binding domain comprises a HCVR and a LCVR, wherein the HCVR comprises HCDR1, HCDR2, and HCDR3, and wherein the LCVR comprises LCDR1, LCDR2, and LCDR3, wherein HCDR1 comprises an amino acid sequence given by SEQ ID NO: 11, HCDR2 comprises an amino acid sequence given by SEQ ID NO: 12, HCDR3 comprises an amino acid sequence given by SEQ ID NO: 13, LCDR1 comprises an amino acid sequence given by SEQ ID NO: 14, LCDR2 comprises an amino acid sequence given by SEQ ID NO: 15, and LCDR3 comprises an amino acid sequence given by SEQ ID NO: 16. In certain embodiments, the HCVR comprises an amino acid sequence given by SEQ ID NO: 17. In certain embodiments, the LCVR comprises an amino acid sequence given by SEQ ID NO: 18. In particular embodiments, the multispecific molecule of the present invention is catabolic.

The present invention provides an antibody that specifically binds ASGR1, comprising a HCVR and a LCVR, wherein the HCVR comprises HCDR1, HCDR2, and HCDR3, and wherein the LCVR comprises LCDR1, LCDR2, and LCDR3, wherein HCDR1 comprises an amino acid sequence given by SEQ ID NO: 27, HCDR2 comprises an amino acid sequence given by SEQ ID NO: 28, HCDR3 comprises an amino acid sequence given by SEQ ID NO: 29, LCDR1 comprises an amino acid sequence given by SEQ ID NO: 30, LCDR2 comprises an amino acid sequence given by SEQ ID NO: 31, and LCDR3 comprises an amino acid sequence given by SEQ ID NO: 32. In certain embodiments, the HCVR comprises an amino acid sequence given by SEQ ID NO: 35. In certain embodiments, the LCVR comprises an amino acid sequence given by SEQ ID NO: 36. In particular embodiments, the antibody of the present invention is non-catabolic.

In certain embodiments, the multispecific molecule of the present invention comprises a binding domain that specifically binds ASGR1, and wherein said binding domain comprises a HCVR and a LCVR, wherein the HCVR comprises HCDR1, HCDR2, and HCDR3, and wherein the LCVR comprises LCDR1, LCDR2, and LCDR3, wherein HCDR1 comprises an amino acid sequence given by SEQ ID NO: 27, HCDR2 comprises an amino acid sequence given by SEQ ID NO: 28, HCDR3 comprises an amino acid sequence given by SEQ ID NO: 29, LCDR1 comprises an amino acid sequence given by SEQ ID NO: 30, LCDR2 comprises an amino acid sequence given by SEQ ID NO: 31, and LCDR3 comprises an amino acid sequence given by SEQ ID NO: 32. In certain embodiments, the HCVR comprises an amino acid sequence given by SEQ ID NO: 35. In certain embodiments, the LCVR comprises an amino acid sequence given by SEQ ID NO: 36. In particular embodiments, the multispecific molecule of the present invention is non-catabolic.

In another aspect, the present invention is directed to an antibody that specifically binds ASGR1, comprising a HCVR and a LCVR, wherein the HCVR comprises HCDR1, HCDR2, and HCDR3, and wherein the LCVR comprises LCDR1, LCDR2, and LCDR3, wherein HCDR1 comprises an amino acid sequence given by SEQ ID NO: 38, HCDR2 comprises an amino acid sequence given by SEQ ID NO: 39, HCDR3 comprises an amino acid sequence given by SEQ ID NO: 40, LCDR1 comprises an amino acid sequence given by SEQ ID NO: 41, LCDR2 comprises an amino acid sequence given by SEQ ID NO: 42, and LCDR3 comprises an amino acid sequence given by SEQ ID NO: 43. In certain embodiments, the HCVR comprises an amino acid sequence given by SEQ ID NO: 44. In certain embodiments, the LCVR comprises an amino acid sequence given by SEQ ID NO: 45. In particular embodiments, the antibody of the present invention is catabolic.

In certain embodiments, the multispecific molecule of the present invention comprises a binding domain that specifically binds ASGR1, and wherein said binding domain comprises a HCVR and a LCVR, wherein the HCVR comprises HCDR1, HCDR2, and HCDR3, and wherein the LCVR comprises LCDR1, LCDR2, and LCDR3, wherein HCDR1 comprises an amino acid sequence given by SEQ ID NO: 38, HCDR2 comprises an amino acid sequence given by SEQ ID NO: 39, HCDR3 comprises an amino acid sequence given by SEQ ID NO: 40, LCDR1 comprises an amino acid sequence given by SEQ ID NO: 41, LCDR2 comprises an amino acid sequence given by SEQ ID NO: 42, and LCDR3 comprises an amino acid sequence given by SEQ ID NO: 43. In certain embodiments, the HCVR comprises an amino acid sequence given by SEQ ID NO: 44. In certain embodiments, the LCVR comprises an amino acid sequence given by SEQ ID NO: 45. In particular embodiments, the multispecific molecule of the present invention is catabolic.

The present invention provides an antibody that specifically binds IgG, comprising a HCVR and a LCVR, wherein the HCVR comprises HCDR1, HCDR2, and HCDR3, and wherein the LCVR comprises LCDR1, LCDR2, and LCDR3, wherein HCDR1 comprises an amino acid sequence given by SEQ ID NO: 21, HCDR2 comprises an amino acid sequence given by SEQ ID NO: 22, HCDR3 comprises an amino acid sequence given by SEQ ID NO: 23, LCDR1 comprises an amino acid sequence given by SEQ ID NO: 24, LCDR2 comprises an amino acid sequence given by SEQ ID NO: 25, and LCDR3 comprises an amino acid sequence given by SEQ ID NO: 26. In certain embodiments, the HCVR comprises an amino acid sequence given by SEQ ID NO: 33. In certain embodiments, the LCVR comprises an amino acid sequence given by SEQ ID NO: 34. In certain embodiments, the HC comprises an amino acid sequence given by SEQ ID NO: 51. In certain embodiments, the LC comprises an amino acid sequence given by SEQ ID NO: 52.

In certain embodiments, the multispecific molecule of the present invention comprises a binding domain that specifically binds IgG, and wherein said binding domain comprises a HCVR and a LCVR, wherein the HCVR comprises HCDR1, HCDR2, and HCDR3, and wherein the LCVR comprises LCDR1, LCDR2, and LCDR3, wherein HCDR1 comprises an amino acid sequence given by SEQ ID NO: 21, HCDR2 comprises an amino acid sequence given by SEQ ID NO: 22, HCDR3 comprises an amino acid sequence given by SEQ ID NO: 23, LCDR1 comprises an amino acid sequence given by SEQ ID NO: 24, LCDR2 comprises an amino acid sequence given by SEQ ID NO: 25, and LCDR3 comprises an amino acid sequence given by SEQ ID NO: 26. In certain embodiments, the HCVR comprises an amino acid sequence given by SEQ ID NO: 33. In certain embodiments, the LCVR comprises an amino acid sequence given by SEQ ID NO: 34.

In certain embodiments, the multispecific molecule of the present invention comprises a binding domain that specifically binds ASGR1 and a binding domain that specifically binds immunoglobulin. In certain embodiments, the binding domain that specifically binds immunoglobulin specifically binds IgG, IgM, IgA, IgD, or IgE. In particular embodiments, the binding domain specifically binds IgG. In other particular embodiments, the binding domain specifically binds IgA. In certain such embodiments, the binding domain that specifically binds ASGR1 and the binding domain that specifically bind IgG are of the present invention. In particular embodiments, the binding domain that specifically binds ASGR1 and/or immunoglobulin is an scFv, scFab, Fab′, and/or Fab.

In certain embodiments, the multispecific molecule of the present invention comprises an amino acid sequence given by SEQ ID NO: 37.

In certain embodiments, the multispecific molecule of the present invention comprises an amino acid sequence given by SEQ ID NO: 46.

In certain embodiments, the multispecific molecule of the present invention comprises an amino acid sequence given by SEQ ID NO: 75.

In certain embodiments, the multispecific molecule of the present invention comprises a HCDR1 comprising SEQ ID NO: 59, HCDR2 comprising SEQ ID NO: 60, HCDR3 comprising SEQ ID NO: 61, LCDR1 comprising SEQ ID NO: 62, LCDR2 comprising SEQ ID NO: 63, and LCDR3 comprising SEQ ID NO: 64. In certain embodiments, the multispecific molecule of the present invention comprises an HCVR comprising SEQ ID NO: 71 and an LCVR comprising SEQ ID NO: 72. In certain embodiments, the multispecific molecule of the present invention comprises a HC comprising SEQ ID NO: 53 and a LC comprising SEQ ID NO: 54. In certain embodiments, the multispecific molecule is an antibody. In certain embodiments, the multispecific molecule is an scFab. In certain embodiments, the multispecific molecule is an scFv. In certain embodiments, the multispecific molecule further comprises a binding arm that binds a recycling target. In certain embodiments, the recycling target is ASGR1.

In certain embodiments, the multispecific molecule of the present invention comprises a HCDR1 comprising SEQ ID NO: 65, HCDR2 comprising SEQ ID NO: 66, HCDR3 comprising SEQ ID NO: 67, LCDR1 comprising SEQ ID NO: 62, LCDR2 comprising SEQ ID NO: 63, and LCDR3 comprising SEQ ID NO: 64. In certain embodiments, the multispecific molecule of the present invention comprises an HCVR comprising SEQ ID NO: 73 and an LCVR comprising SEQ ID NO: 72. In certain embodiments, the multispecific molecule of the present invention comprises a HC comprising SEQ ID NO: 55 and a LC comprising SEQ ID NO: 56. In certain embodiments, the multispecific molecule is an antibody. In certain embodiments, the multispecific molecule is an scFab. In certain embodiments, the multispecific molecule is an scFv. In certain embodiments, the multispecific molecule further comprises a binding arm that binds a recycling target. In certain embodiments, the recycling target is ASGR1.

In certain embodiments, the multispecific molecule of the present invention comprises a HCDR1 comprising SEQ ID NO: 68, HCDR2 comprising SEQ ID NO: 69, HCDR3 comprising SEQ ID NO: 70, LCDR1 comprising SEQ ID NO: 62, LCDR2 comprising SEQ ID NO: 63, and LCDR3 comprising SEQ ID NO: 64. In certain embodiments, the multispecific molecule of the present invention comprises an HCVR comprising SEQ ID NO: 74 and an LCVR comprising SEQ ID NO: 72. In certain embodiments, the multispecific molecule of the present invention comprises a HC comprising SEQ ID NO: 57 and a LC comprising SEQ ID NO: 58. In certain embodiments, the multispecific molecule is an antibody. In certain embodiments, the multispecific molecule is an scFab. In certain embodiments, the multispecific molecule is an scFv. In certain embodiments, the multispecific molecule further comprises a binding arm that binds a recycling target. In certain embodiments, the recycling target is ASGR1.

Also provided herein are one or more nucleic acid sequences encoding an antibody of the present invention or a multispecific molecule of the present invention. In certain embodiments, the present invention provides a DNA molecule comprising a polynucleotide that encodes a HC or HCVR of an antibody or multispecific molecule of the present invention. The present invention also provides a DNA molecule comprising a polynucleotide that encodes a LC or LCVR of an antibody or multispecific molecule of the present invention. The present invention also provides a DNA molecule comprising a polynucleotide that encodes both a LC or LCVR of an antibody or multispecific molecule of the present invention and a HC or HCVR of an antibody or multispecific molecule of the present invention.

The present invention further provides a mammalian cell transformed with a DNA molecule of the present invention, wherein the transformed mammalian cell is capable of expressing an antibody or multispecific molecule of the present invention.

The present invention also provides a process for producing an antibody or multispecific molecule of the present invention, wherein the process comprises cultivating a mammalian cell under conditions such that the antibody or multispecific molecule is expressed and recovering the expressed antibody or multispecific molecule. In an embodiment, the mammalian cell is transformed with a DNA molecule of the present invention, wherein the transformed mammalian cell is capable of expressing an antibody or multispecific molecule of the present invention. The present invention also provides an antibody or multispecific molecule obtainable by the process.

The present invention provides a multispecific molecule of the present invention for use in therapy.

The present invention provides a multispecific molecule of the present invention for use in treating autoantibody-induced disease.

The present invention provides a multispecific molecule of the present invention for the manufacture of a medicament for the treatment of autoantibody-induced disease.

In certain embodiments, the autoantibody-induced disease is selected from the group consisting of myasthenia gravis, Guillain-Barre syndrome, epilepsy, autoimmune limbic encephalitis, spinal cord injury, pediatric autoimmune neuropsychiatric disorders associated with streptococcal infection, neuromyotonia, morvan syndrome, multiple sclerosis, pemphigus vulgaris, pemphigus foliaceus, bullous pemphigoid, epidermosysis bullosa acquisita, pemphigoig gestationis, mucous membrane pemphigoid, licen sclerosus, antiphospholipid syndrome, relapsing polychondritis, autoimmune anemia, idiopathic trombocytic purpura, autoimmune Grave's disease, dilated cardiomyopathy, vasculitis, goodpasture's syndrome, idiopathic membranous nephropathy, rheumatoid arthritis, and systemic lupus erythematosus.

The present invention provides a method of treating a patient having at least one autoantibody-induced disease comprising administering to the patient an effective amount of a multispecific molecule of the present invention. In certain embodiments, the patient has at least one autoantibody-induced disease. In certain embodiments, the patient has at least one of myasthenia gravis, Guillain-Barre syndrome, epilepsy, autoimmune limbic encephalitis, spinal cord injury, pediatric autoimmune neuropsychiatric disorders associated with streptococcal infection, neuromyotonia, morvan syndrome, multiple sclerosis, pemphigus vulgaris, pemphigus foliaceus, bullous pemphigoid, epidermosysis bullosa acquisita, pemphigoig gestationis, mucous membrane pemphigoid, licen sclerosus, antiphospholipid syndrome, relapsing polychondritis, autoimmune anemia, idiopathic trombocytic purpura, autoimmune Grave's disease, dilated cardiomyopathy, vasculitis, goodpasture's syndrome, idiopathic membranous nephropathy, rheumatoid arthritis, and systemic lupus erythematosus.

The present invention also provides a pharmaceutical composition comprising a multispecific molecule of the present invention and one or more pharmaceutically acceptable carriers, diluents, or excipients.

### BRIEF DESCRIPTION OF THE DRAWINGS

FIGS. 1A, 1B, and 1C depict differential clearance of Non-CAT and CAT anti-ASGR1 antibodies in human FcRn Tg mice. Mice were intravenously administered with non-catabolic antibody (FIG. 1A) or catabolic antibody (FIG. 1B), and total antibody concentrations were measure over time. Individual measurements were marked by (▾) for 0.3 mg/kg, (▴) 3 mg/kg, (▪) 10 mg/kg, and (●) 30 mg/kg dose of antibodies. FIG. 1C depicts the data from FIG. 1B from time zero to 24 hours.

FIG. 2 depicts simultaneous binding of ASGR1 and IVIg to immobilized multispecific molecule of the present invention.

FIG. 3 depicts rapid depletion of exogenously administered human IgGs in mice post administration of catabolic bispecific scFv molecule of the present invention. Mice were administered human IgGs followed by administration of either bispecific scFv molecule of the present invention (a bispecific scFv) or PBS. Concentration time profile of human IgGs in mouse serum was plotted by quantifying human IgG concentration through ELISA post administration of either bispecific scFv molecules of the present invention or PBS.

FIG. 4 depicts rapid depletion of exogenously administered human IgGs in cynomolgus monkeys post administration of catabolic bispecific scFv molecule of the present invention. Concentration time profile of human IgGs in cynomolgus monkey serum was plotted by quantifying human IgG concentration through ELISA post administration of either bispecific scFv molecules of the present invention or PBS.

FIG. 5A and FIG. 5B depict bispecific scFv molecules of the present inventions binding to ASGR1 in a catabolic and non-catabolic manner are equal potent in depleting huIgGs in-vivo. Seventy-two hours post administration of huIVIg, animals were administered with either PBS, or catabolic bispecific scFv molecules of the present invention or non-catabolic bispecific scFv molecules of the present invention. Normalized serum (FIG. 5A) and whole-body (FIG. 5B) radioactive counts of mice administered with I-125 labeled huIVIg are shown.

FIG. 6 depicts a bispecific scFv molecule of the present invention that drives IgGs to liver for rapid catabolization. Mice were administered IVIg labeled with I-125 (left column) or In-111 (right column), and post 72 hours animals were administered with either catabolic bispecific scFv molecule of the present invention or PBS. Animals were perfused at organs were harvested at 3 hours, 24 hours, or 96 hours post administration of clearance agents and their radioactive counts were measured and plotted.

### DETAILED DESCRIPTION

The present invention provides multispecific molecules that bind immunoglobulin and a recycling target. In certain embodiments, the multispecific molecule comprises a first binding domain that specifically binds immunoglobulin and a second binding domain that specifically binds a recycling target. Multispecific molecule bound to immunoglobulin and the recycling target is internalized into the cell, after which the immunoglobulin is degraded in the lysosome. In certain embodiments, the recycling target remains bound to the multispecific molecule, and recycles back to the cell surface. Said multispecific molecule then is able to bind another immunoglobulin and internalize it for degradation. In certain embodiments, the recycling target disengages the multispecific molecule inside the cell before recycling back to the cell surface.

Multispecific molecules of the present invention comprise at least two binding domains. Said binding domain is an antigen-binding portion of an antibody, or a binding domain derived from antigen-binding portion of an antibody. Any binding domain is thought to be suitable, provided the binding domain specifically binds to either immunoglobulin or recycling target. In certain embodiments, the binding domain is devoid of an Fc region. Examples of binding domains include scFv, Fab, scFab, Fab′, Fv, and dsFv. Multispecific molecules comprising binding domains may be in a format such as F(ab′)₂, (scFv-Zip)₂, (scFv)₂ (e.g. BiTE® molecule), diabody, scDb, and tandem diabody. Also envisioned are multispecific molecules comprising binding domains derived from camelid antibodies. In certain embodiments, said multispecific molecules comprising binding domains derived from camelid antibodies comprise at least one camelid VH and at least one camelid VL, at least two camelid VHs, or at least two camelid VLs. In certain embodiments, said molecules further comprise a half-life extending (HLE) moiety. Also envisioned are UniDab® molecules. Binding domains and multispecific molecules of the present invention can be produced according to well-known methods (see e.g. Kipriyanov S. M. (2003); Recombinant Antibodies for Cancer Therapy; 207(3-26); Janssens et al, Oct. 10, 2006; PNAS; 103(41)15130-15135).

Nonlimiting examples of HLE moieties include an Fc polypeptide, a single-chain Fc polypeptide (scFc), albumin, an albumin fragment, a moiety that binds to albumin or to the neonatal Fc receptor (FcRn), a derivative of fibronectin that has been engineered to bind albumin or a fragment thereof, a peptide, a single domain protein fragment, or other polypeptide that can increase serum half-life. In other embodiments, a half-life-extending moiety can be a non-polypeptide molecule such as, for example, polyethylene glycol (PEG). In certain embodiments, the HLE is a single-chain Fc (“scFc”).

The present invention provides bispecific scFv molecules that bind immunoglobulin and a recycling target. Said bispecific scFv molecules comprise an scFv that specifically binds immunoglobulin (“first scFv”) connected via a linker to an scFv that specifically binds a recycling target (“second scFv”).

An “recycling target”, as used herein, refers to a protein that is internalized into the cell from the cell surface and then brought back to the cell surface. Hence, a recycling target is one that is recycled to the cell surface. Internalization of the protein may occur via endocytosis, which can occur by various mechanisms. In a general sense, endocytosis begins with the formation of endocytic vesicles carrying endocytosed cargo into the cell, which cargo is then delivered to the early endosome. Cargo can then go to the late endosome and lysosome for degradation, to the trans-Golgi network (TGN) or to recycling endosomal carriers that bring the cargo back to the plasma membrane. In this context as it relates to the present invention, cargo refers to a recycling target that is bound by a multispecific molecule of the present invention, which multispecific molecule is also bound to immunoglobulin. Recycling targets are receptors that can rapidly internalize into the cell (endosome) and recycle back to the cell surface. Examples of a recycling target include, but are not limited to, Asialoglycoprotein Receptor 1 (ASGR1), transferrin receptor, and mannose 6 phosphate receptor.

ASGR1 is a membrane-bound receptor expressed in hepatocytes that is made of the ASGPR1 and ASGPR2 subunits. ASGR1 removes desialylated glycoproteins from circulation via receptor-mediated endocytosis. ASGR1 has been shown to have a receptor recycling time of approximately 10-15 minutes in human cells. ASGR1 has been used for liver-specific delivery of compounds including small molecules (see e.g. Willoughby et al., Mol Ther. 2018 Jan. 3; 26(1): 105-114). In certain embodiments, ASGR1 is the recycling target. ASGR1 is highly expressed on cell surface of hepatocytes, it has very fast internalization and recycling rates, and it can help in depleting large loads of antigens. As ASGR1 is expressed in the liver and catabolization of antigen happens in liver, the expected toxicity using ASGR1 as a recycling target is expected to be acceptable.

The early endosome has a pH of about 6.5 to 6.0, the late endosome has a pH of about 5.5, and the lysosome has a pH of about 4.0. Multispecific molecules of the present invention result in depletion (or clearance) of immunoglobulin (Ig) in the lysosome. Depletion of immunoglobulin can be measured by assays known in the art, including immunoassays, radioactivity in blood, and flow cytometry assays.

In certain embodiments, the multispecific molecule is said to be catabolic (“CAT”). Catabolic molecules are molecules that either disengage from the recycling target in the endosome (“recycling target catabolic molecule”) and/or disengage from the Ig in the endosome. In certain embodiments, a recycling target catabolic molecule remains bound to Ig (e.g. a molecule that binds in a catabolic manner to ASGR1 and in a non-catabolic manner to Ig). In certain embodiments, a recycling target catabolic molecule also dissociates from the Ig (e.g. a molecule that binds in a catabolic manner to ASGR1 and in a catabolic manner to Ig). In certain embodiments, a catabolic molecule dissociates from the Ig but remains bound to the recycling target (e.g. a molecule that binds in a non-catabolic manner to ASGR1 and in a catabolic manner to Ig). Upon disengagement from the recycling target and/or Ig, the Ig is degraded in the lysosome. In certain embodiments, the recycling target recycles back to the cell surface. In certain such embodiments in which the multispecific molecule binds the recycling target in a non-catabolic manner and Ig in a catabolic manner, and the multispecific molecule is recycled with the recycling target, these catabolic molecules are expected to be given at a lower dose in a patient to treat autoantibody-induced disease.

Catabolic molecules are sensitive to the low pH in the endosome, which low pH results in dissociation of the multispecific molecule and Ig from the recycling target (or results in dissociation of Ig from the multispecific molecule). Catabolic molecules may demonstrate, for example, reduced binding to ASGR1 (and/or Ig) in conditions of pH 6.0 and 2 μM calcium chloride. Molecules exhibiting high affinity to the recycling target and/or Ig at neutral pH/high calcium concentration, but no detectable binding at acidic pH/low calcium concentration enable faster degradation (catabolization) of Ig, and are so termed catabolic.

In certain embodiments, the multispecific molecule is said to be non-catabolic (“non-CAT”). A non-CAT molecule is a multispecific molecule that remains bound to the recycling target and to the Ig, and which complex (non-CAT molecule, recycling target, and Ig) recycles back to the cell surface. In contrast to catabolic molecules, non-catabolic molecules are pH-insensitive, and therefore do not dissociate from the recycling target or Ig in the endosome. Non-catabolic molecules may demonstrate, for example, similar binding to ASGR1 and Ig in conditions of pH 6.0 and 2 μM calcium chloride compared to conditions of neutral pH (pH 7.4) and 2 mM calcium chloride. In certain embodiments, the recycling target and/or non-catabolic molecule and/or Ig is degraded.

According to certain embodiments of the present invention, the binding domain that specifically binds Ig (e.g. IgG) is non-catabolic, and the binding domain that specifically binds ASGR1 is catabolic. In other embodiments, the binding domain that specifically binds Ig (e.g. IgG) is catabolic, and the binding domain that specifically binds ASGR1 is non-catabolic. In other embodiments, the binding domain that specifically binds Ig (e.g. IgG) is non-catabolic, and the binding domain that specifically binds ASGR1 is non-catabolic.

In certain embodiments, a bispecific scFv molecules of the present invention comprises an scFv that specifically binds a recycling target, and an scFv that specifically binds immunoglobulin. A bispecific scFv molecule of the present invention may be a single-chain polypeptide that comprises a first scFv-linker-second scFv. An scFv, or single-chain variable fragment, is made of the variable domains of an antibody heavy chain and light chain that may be linked together by a short peptide linker. For example, (G₄S)₃ linker may be used, at any number of repeats, such as one to four. The orientation from the N-terminus to the C-terminus of each scFv may be VL-linker-VH or VH-linker-VL.

According to one embodiment of the invention, a multispecific molecule of the invention is a single chain molecule. Although the two domains of the binding domain, VL and VH, are coded for by separate genes, they can be joined, using recombinant methods, by an artificial linker—as described hereinbefore—that enables them to be made as a single protein chain in which the VL and VH regions pair to form a monovalent molecule; see e.g., Huston et al. (1988) Proc. Natl. Acad. Sci USA 85:5879-5883).

An example of a bispecific scFv molecule that can be engineered from an scFab that binds mIgG2a in a catabolic manner and a molecule that binds ASGR1 in a non-catabolic manner is SEQ ID NO: 75. Cysteine(s) may be further introduced to improve stability (see e.g. Reiter et al., Biochemistry 1994, 33, 5451-5459) and are not expected to alter catabolic properties of the molecule.

The conversion of an scFab molecule to a bispecific scFv molecule is not expected to alter the catabolic properties of the molecule.

Binding domains are obtained using conventional techniques known to those with skill in the art, and the binding domains are evaluated for function in the same manner as are full-length antibodies or IgGs. An scFv for example is hence a fusion protein of the variable region of the heavy chain (VH) and of the light chain (VL) of immunoglobulins, usually connected with a short linker peptide. The linker is usually rich in glycine for flexibility, as well as serine or also threonine for solubility, and can either connect the N-terminus of the VH with the C-terminus of the VL, or vice versa. This protein retains the specificity of the original immunoglobulin, despite removal of the constant regions and introduction of the linker.

Bispecific single chain molecules are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Loffler, Blood, (2000), 95, 6, 2098-2103, Bruhl, Immunol., (2001), 166, 2420-2426, Kipriyanov, J. Mol. Biol., (1999), 293, 41-56. Techniques described for producing single chain antibody constructs (see, inter alia, U.S. Pat. No. 4,946,778, Kontermann and Diibel (2010), loc. cit. and Little (2009), loc. cit.) can be adapted to produce single chain antibody constructs specifically recognizing (an) elected target(s).

Bivalent (also called divalent) or bispecific single-chain variable fragments (bi-scFvs or di-scFvs) having the format (scFv)2 can be engineered by linking two scFv molecules (e.g. with linkers as described hereinbefore). The linking can be done by producing a single polypeptide chain with two VH regions and two VL regions, yielding tandem scFvs (see e.g. Kufer P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. In this case, the VH and the VL of a binding domain (binding either to immunoglobulin or recycling target) are not directly connected via a peptide linker. Thus, the VH of the immunoglobulin binding domain may e.g. be fused to the VL of the recycling target binding domain via a peptide linker, and the VH of the recycling target binding domain is fused to the VL of the immunoglobulin binding domain via such peptide linker. This type is known as diabodies (see e.g. Hollinger, Philipp el al., (July 1993) Proceedings of the National Academy of Sciences of the United States of America 90 (14): 6444-8.).

In certain embodiments, multispecific molecules of the present invention, such as bispecific scFv molecules, comprise two binding domains (e.g. scFvs) connected to one another by a linker. The linker connecting the two binding domains can be a helical linker or a flexible linker, for example. The term “peptide linker” comprises in accordance with the present invention an amino acid sequence by which the amino acid sequences of one (variable and/or binding) domain and another (variable and/or binding) domain of the multispecific molecule of the invention are linked with each other. Among the suitable peptide linkers are those described in U.S. Pat. Nos. 4,751,180 and 4,935,233 or WO 88/09344.

An example of a peptide linker connecting the two binding domains is a SG₄S linker. A multispecific molecule of the present invention may have one, two, three, four, five, or six repeats of the SG₄S linker. For example two SG₄S repeats would be binding domain (e.g. scFv)-SGGGGSSGGGGS-binding domain (e.g. scFv) (SGGGGSSGGGGS given by SEQ ID NO: 98). In certain embodiments, the multispecific molecules of the present invention have one SG₄S as a linker. In other embodiments, the linker comprises two, three, or four SG₄S repeats. In other embodiments, the linker comprises five or six SG₄S repeats. In other embodiments, the linker comprises seven or more SG₄S repeats, as long as the multispecific molecule is able to be expressed and purified. Other examples of linkers include linkers comprising a sequence selected from the group consisting of (Gly₃Ser)₃ (SEQ ID NO: 76), (Gly₄Ser)₃ (SEQ ID NO: 77), (Gly₃Ser)₄ (SEQ ID NO: 78), (Gly₄Ser)₄ (SEQ ID NO: 79), (Gly₃Ser)₅ (SEQ ID NO: 80), (Gly₄Ser)₅ (SEQ ID NO: 81), (Gly₃Ser)₆ (SEQ ID NO: 82), (Gly₄Ser)₆ (SEQ ID NO: 83), GSADDAKKDAAKKDAAKKDDAKKDDAGS (SEQ ID NO: 84), GSADDAKKDAAKKDAAKKDDAKKDDAKKDAGS (SEQ ID NO: 85), (Gly₃Gln)₂ (SEQ ID NO: 86), (Gly₄Gln)₂ (SEQ ID NO: 87), (Gly₃Gln)₃ (SEQ ID NO: 88), (Gly₄Gln)₃ (SEQ ID NO: 89), (Gly₃Gln)₄ (SEQ ID NO: 90), (Gly₄Gln)₄ (SEQ ID NO: 91), (Gly₃Gln)₅ (SEQ ID NO: 92), (Gly₄Gln)₅ (SEQ ID NO: 93), (Gly₃Gln)₆ (SEQ ID NO: 94), (Gly₄Gln)₆ (SEQ ID NO: 95), (Gly₃Ser)₂ (SEQ ID NO: 96), and (Gly₄Ser)₂ (SEQ ID NO: 97).

As used herein, an “antibody” is an immunoglobulin molecule comprising 2 heavy chains (HCs) and 2 light chains (LCs) interconnected by disulfide bonds. The amino terminal portion of each LC and HC includes a variable region of about 100-120 amino acids primarily responsible for antigen recognition via the CDRs contained therein. The CDRs are interspersed with regions that are more conserved, termed framework regions (“FR”). Each light chain variable region (LCVR) and heavy chain variable region (HCVR) is composed of 3 CDRs and 4 FRs, arranged from amino-terminus to carboxy-terminus in the following order: FR1, CDR1, FR2, CDR2, FR3, CDR3, FR4. The 3 CDRs of the LC are referred to as “LCDR1, LCDR2, and LCDR3,” and the 3 CDRs of the HC are referred to as “HCDR1, HCDR2, and HCDR3.”

The CDRs contain most of the residues which form specific interactions with the antigen. The functional ability of an antibody to bind a particular antigen is, thus, largely influenced by the amino acid residues within the six CDRs.

The binding domain characterized in connection with the present invention is a domain which specifically binds to/interacts with/recognizes a given target epitope or a given target side on the target molecules (antigens), here: immunoglobulin or recycling target. The structure and function of the first binding domain and the second binding domain are based on or derived from the structure and/or function of an antibody, more particularly, they are drawn from or derived from the variable heavy chain (VH) and variable light chain (VL) domains of an antibody. In certain embodiments, the binding domain is characterized by the presence of three light chain CDRs (i.e. CDR1, CDR2 and CDR3 of the VL region) and three heavy chain CDRs (i.e. CDR1, CDR2 and CDR3 of the VH region). Assignment of amino acids to CDR domains within the LCVR and HCVR regions of the antibodies of the present invention described herein is based on the known numbering convention termed AHo (A. Honegger & A. Pluckthun. “Yet another numbering scheme for immunoglobulin variable domains: An automatic modeling and analysis tool”. J. Mol. Biol, 309 (2001) 657-670). It is understood that other numbering conventions may also be used, such as, for example, Kabat numbering convention (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)); Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), and/or North (North et al., “A New Clustering of Antibody CDR Loop Conformations”, Journal of Molecular Biology, 406, 228-256 (2011)).

As used herein, an antigen binding domain (and/or a multispecific molecule) is said to “specifically bind” to its antigen when the binding domain and/or a multispecific molecule binds its antigen with a dissociation constant (KD) of ≤10⁻⁶ M as measured via a surface plasma resonance technique (e.g., BIACore, GE-Healthcare Uppsala, Sweden) or Kinetic Exclusion Assay (KinExA, Sapidyne, Boise, Idaho).

It is envisaged that the binding domain of the present invention is produced by or obtainable by phage-display or library screening methods or by grafting CDR sequences from a pre-existing monoclonal antibody into a scaffold. Catabolic molecules may be obtained by screening molecules that yield catabolic activity as described herein. Engineering (e.g. in CDR(s)) may also be employed to obtain catabolic molecules. Engineering by phage display and/or histidine scanning may also be employed to introduce histidine residue(s) in the CDRs. Histidine has a PK around 6.5 and hence binding would be disrupted in acidic conditions in the endosome, yielding a catabolic molecule.

In the context of an antibody, the present invention contemplates antibodies that may have clipping of the C-terminal lysine or cysteine residue of the HC.

In the context of an antibody or binding domain of the present invention, the N-terminal glutamine and/or the N-terminal glutamic acid may be converted to pyroglutamic acid.

Multispecific molecules according to the present invention are envisioned to have a format that does not result in self-binding. For example a multispecific molecule engineered to bind IgG and a recycling target should be devoid of an Fc region. In certain embodiments, multispecific molecules according to the present invention preferably demonstrate monovalent binding to immunoglobulin, as immunoglobulin cross-linking can activate the immune system resulting in side effects such as anaphylaxis.

As used herein, “multispecific” refers to a molecule that comprises at least a first binding domain and a second binding domain, wherein the first binding domain specifically binds to one antigen (target), and the second binding domain specifically binds to another antigen (target). Accordingly, multispecific molecules according to the invention comprise specificities for at least two different antigens or targets. In certain embodiments, a multispecific molecule comprises no less than two, and no more than two, binding domains (each binding domain specifically binds a different target, e.g. immunoglobulin and recycling target), and said multispecific molecule may be referred to as a “bispecific molecule.” A “bispecific scFv molecule,” as used herein, therefore refers to a multispecific molecule that binds two different targets (antigens), such as immunoglobulin and a recycling target (e.g. ASGR1), and comprises two scFvs (binding domains), wherein one scFv specifically binds immunoglobulin, and the other scFv specifically binds the recycling target.

Multispecific molecules of the present invention bind and deplete immunoglobulin. Depletion of immunoglobulin is thought to be beneficial in the treatment of patients having antibody-mediated autoimmune disease (also referred to as autoantibody-induced disease). As used herein, a “patient” refers to a human. Autoantibody-induced disease result from the body's immune system not being able to discriminate between self and non-self antigens, resulting in the immune system attacking normal parts on the body potentially resulting in damage and/or disease. For example, the immune system may begin producing antibodies that attack the body's own tissues. Examples of treatments for autoimmune disease include anti-inflammatory drugs, corticosteroids, pain-killing medication, immunosuppressant drugs, physical therapy, surgery, high dose immunosuppression, and disease-specific treatments. Other therapies for use in autoimmune disease are plasmapheresis, i.v. Ig (IVIg) administration, and immunoadsorption; however these are associated with high cost of treatment and/or side effects. In the clinic, ABDEG® (“antibodies that enhance IgG degradation”) molecules (antibody-based FcRn inhibitor) have been shown to deplete human IgGs by ˜50-70% within 2-3 weeks after administration.

The molecules of the present invention can readily be produced in mammalian cells, non-limiting examples of which includes CHO, NSO, HEK293 or COS cells. The host cells are cultured using techniques well known in the art.

In certain embodiments, the present invention provides vectors comprising a nucleic acid encoding a polypeptide of the invention or a portion thereof. Examples of vectors include, but are not limited to, plasmids, viral vectors, non-episomal mammalian vectors and expression vectors, for example, recombinant expression vectors. Vectors containing the polynucleotide sequences of interest (e.g., the polynucleotides encoding the polypeptides of the molecule and expression control sequences) can be transferred into the host cell by well-known methods, which vary depending on the type of cellular host. Examples of vectors include, but are not limited to, plasmids, viral vectors, non-episomal mammalian vectors and expression vectors, for example, recombinant expression vectors.

The recombinant expression vectors of the invention can comprise a nucleic acid of the invention in a form suitable for expression of the nucleic acid in a host cell. The recombinant expression vectors include one or more regulatory sequences, selected on the basis of the host cells to be used for expression, which is operably linked to the nucleic acid sequence to be expressed. Regulatory sequences include those that direct constitutive expression of a nucleotide sequence in many types of host cells (e.g., SV40 early gene enhancer, Rous sarcoma virus promoter and cytomegalovirus promoter), those that direct expression of the nucleotide sequence only in certain host cells (e.g., tissue-specific regulatory sequences, see Voss et al., 1986, Trends Biochem. Sci. 11:287, Maniatis et al., 1987, Science 236:1237, incorporated by reference herein in their entireties), and those that direct inducible expression of a nucleotide sequence in response to particular treatment or condition (e.g., the metallothionin promoter in mammalian cells and the tet-responsive and/or streptomycin responsive promoter in both prokaryotic and eukaryotic systems (see id.). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

In certain embodiments, the present invention provides host cells into which a recombinant expression vector of the invention has been introduced. A host cell can be any prokaryotic cell or eukaryotic cell. Prokaryotic host cells include gram negative or gram positive organisms, for example E. coli or bacilli. Higher eukaryotic cells include insect cells, yeast cells, and established cell lines of mammalian origin. Examples of suitable mammalian host cell lines include Chinese hamster ovary (CHO) cells or their derivatives such as Veggie CHO and related cell lines which grow in serum-free media (see Rasmussen et al., 1998, Cytotechnology 28:31) or CHO strain DXB-11, which is deficient in DHFR (see Urlaub et al., 1980, Proc. Natl. Acad. Sci. USA 77:4216-20). Additional CHO cell lines include CHO-K1 (ATCC #CCL-61), EM9 (ATCC #CRL-1861), and UV20 (ATCC #CRL-1862). Additional host cells include the COS-7 line of monkey kidney cells (ATCC CRL 1651) (see Gluzman et al., 1981, Cell 23:175), L cells, C127 cells, 3T3 cells (ATCC CCL 163), AM-1/D cells (described in U.S. Pat. No. 6,210,924), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Appropriate cloning and expression vectors for use with bacterial, fungal, yeast, and mammalian cellular hosts are described by Pouwels et al. (Cloning Vectors: A Laboratory Manual, Elsevier, New York, 1985).

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. The leader sequence may comprise an amino acid sequence of SEQ ID NO: 47 (MDMRVPAQLLGLLLLWLRGARC) which is encoded by SEQ ID NO: 48 (atggacatgagagtgcctgcacagctgctgggcctgctgctgctgtggctgagaggcgccagatgc). The leader sequence may comprise an amino acid sequence of SEQ ID NO: 49. (MAWALLLLTLLTQGTGSWA) which is encoded by SEQ ID NO: 50 (atggcctggg ctctgctgct cctcaccctc ctcactcagg gcacagggtc ctgggcc). The present invention contemplates molecule protein sequences without leader sequences.

Various methods of protein purification may be employed to purify proteins, including, but not limited to, antibodies or binding domains, and such methods are known in the art.

The molecules of the invention can be biosynthesized, purified, and formulated for administration by well-known methods. For example, an appropriate host cell, such as HEK 293 or CHO, is either transiently or stably transfected with an expression system for secreting antibodies or binding domains using a predetermined HC:LC or HCVR: LC vector ratio if two vectors are used, or a single vector system encoding both heavy chain and light chain. Vectors suitable for expression and secretion of antibodies or binding domains from these commonly-used host cells are well-known. Following expression and secretion of the antibody or binding domain, the medium is clarified to remove cells and the clarified medium is purified using any of many commonly-used techniques. For example, the medium may be applied to a Protein A or G column that has been equilibrated with a buffer, such as phosphate buffered saline (pH 7.4). The column is washed to remove nonspecific binding components. The bound antibody or binding domain is eluted, for example, by a pH gradient (such as 0.1 M sodium phosphate buffer pH 6.8 to 0.1 M sodium citrate buffer pH 2.5). Antibody or binding domain fractions are detected, such as by SDS-PAGE, and then are pooled. Further purification is optional, depending on the intended use. The antibody or binding domain may be concentrated and/or sterile filtered using common techniques. Other materials than the antibody or binding domain, such as host cell and growth medium components, and soluble aggregates and multimers of the antibody or binding domain, may be effectively reduced or removed by common techniques, including size exclusion, hydrophobic interaction, cation exchange, anion exchange, affinity, or hydroxyapatite chromatography. The purity of the antibody or binding domain after these chromatography steps is typically greater than 95%. The product may be frozen at −70° C. or may be lyophilized.

Vector DNA can be introduced into prokaryotic or eukaryotic cells via conventional transformation or transfection techniques. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Additional selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die), among other methods.

A polynucleotide encoding an amino acid sequence of a molecule of the present invention can be any length as appropriate for the desired use or function, and can comprise one or more additional sequences, for example, regulatory sequences, and/or be part of a larger nucleic acid, for example, a vector. The skilled artisan will appreciate that, due to the degeneracy of the genetic code, each of the polypeptide sequences disclosed herein is encoded by a large number of other nucleic acid sequences. Mutations can also be introduced into a nucleic acid without significantly altering the biological activity of a polypeptide that it encodes. For example, one can make nucleotide substitutions leading to amino acid substitutions at non-essential amino acid residues.

Transformed cells can be cultured under conditions that promote expression of the polypeptide, and the polypeptide recovered by conventional protein purification procedures. Polypeptides contemplated for use herein include substantially homogeneous recombinant mammalian polypeptides substantially free of contaminating endogenous materials. Cells containing the nucleic acid encoding the molecules of the present invention also include hybridomas.

In certain embodiments, a vector comprising a nucleic acid molecule as described herein is provided. In certain embodiments, the invention comprises a host cell comprising a nucleic acid molecule as described herein. In certain embodiments, a nucleic acid molecule encoding a molecule as described herein is provided. In certain embodiments, a pharmaceutical composition comprising at least one molecule described herein is provided.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the molecules included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Immunoglobulins are made by B cells and plasma cells, and are important in the humoral immune responses against bacteria, viruses, fungi, parasites, cellular antigens, chemicals, and synthetic substances. Immunoglobulins can be classified as either IgG, IgM, IgA, IgD, or IgE, based on their heavy chain constant regions. Immunoglobulins are expressed primarily on B cells, after which they are secreted and circulate in blood (see e.g. Hoffman et al., Clin J Am Soc Nephrol. 2016 Jan. 7; 11(1): 137-154). As used herein, the phrase “circulating in blood” refers to immunoglobulins that have been secreted and are circulating in blood.

IgM serves as a first line of defense and provides short-term protection. IgA is also called a secretory antibody and it is secreted via mucous. IgD and IgE make up a relatively small percentage of serum antibodies, although they still play a role in the innate immune system and against parasitic infections, respectively.

IgG is the most common immunoglobulin in the body, making up about 75-80% of antibodies found in blood plasma. IgG is able to activate the complement system, and also has the longest lifespan of immunoglobulins. The long half-life of IgG is due to a recycling pathway involving the neonatal fragment crystallizable receptor (FcRn). Modalities to inhibit FcRn therefore are thought to deplete IgG via lysosomal degradation (see e.g. Hans-Hartmut et al., J Allergy Clin Immunol. 2020 September; 146(3): 479-491). One such antibody-based FcRn inhibitor is referred to as ABDEG (see Challa et al., MAbs. 2013 Sep. 1; 5(5): 655-659). Efgartimgimod, a human IgG1-derived Fc fragment modified using ABDEG technology, demonstrated reduced IgGs in humans (Ulrichts et al., J Clin Invest. 2018; 128(10):4372-438).

Multispecific molecules of the present invention are intended to treat autoantibody-mediated diseases. Autoantibody-mediated diseases include, and are not limited to, myasthenia gravis, Guillain-Barre syndrome, epilepsy, autoimmune limbic encephalitis, spinal cord injury, pediatric autoimmune neuropsychiatric disorders associated with streptococcal infection, neuromyotonia, morvan syndrome, multiple sclerosis, pemphigus vulgaris, pemphigus foliaceus, bullous pemphigoid, epidermosysis bullosa acquisita, pemphigoig gestationis, mucous membrane pemphigoid, licen sclerosus, antiphospholipid syndrome, relapsing polychondritis, autoimmune anemia, idiopathic trombocytic purpura, autoimmune Grave's disease, dilated cardiomyopathy, vasculitis, goodpasture's syndrome, idiopathic membranous nephropathy, rheumatoid arthritis, and systemic lupus erythematosus (see e.g. Wang L et al., J. Internal Medicine, 2015, 278, 369-395; Ludwig R J et al., Front Immunol. 2017, 8, 603; and Pruss H, 2021, Nat. Rev. Immunol, 21(12), 798-813).

Multispecific molecules of the present invention, or a pharmaceutical composition comprising the same, may be administered by parenteral routes, non-limiting examples of which are subcutaneous administration and intravenous administration. Intramuscular, intraarterial, intralesional, and peritoneal bolus injection are other possible routes of administration. Multispecific molecules can also be administered via infusion, for example intravenous or subcutaneous infusion. Multispecific molecules of the present invention may be administered to a patient with pharmaceutically acceptable carriers, diluents, or excipients in single or multiple doses. Optionally, the composition additionally comprises one or more physiologically active agents. Pharmaceutical compositions of the present invention can be prepared by methods well known in the art (e.g., Remington: The Science and Practice of Pharmacy, 22nd ed. (2012), A. Loyd et al., Pharmaceutical Press) and comprise a multispecific molecule, as disclosed herein, and one or more pharmaceutically acceptable carriers, diluents, or excipients.

As used interchangeably herein, “treatment” and/or “treating” and/or “treat” are intended to refer to all processes wherein there may be a slowing, interrupting, arresting, controlling, stopping, or reversing of the progression of the disorders described herein, but does not necessarily indicate a total elimination of all disorder symptoms. Treatment includes administration of a multispecific molecule of the present invention for treatment of a disease or condition in a human that would benefit from activity of a multispecific molecule of the present invention, and includes: (a) inhibiting further progression of the disease; and (b) relieving the disease, i.e., causing regression of the disease or disorder or alleviating symptoms or complications thereof. “Therapy” or “therapeutic,” as used herein, refers to the treatment of a patient having at least one autoantibody-induced disease.

Therapeutically effective amounts (or dose) of a multispecific molecule of the present invention can be administered. As used herein, an “effective amount” means the amount of a multispecific molecule of the present invention or pharmaceutical composition comprising such a multispecific molecule that will elicit the biological or medical response of or desired therapeutic effect on a tissue, system, animal, mammal, or human that is being sought by the researcher, medical doctor, or other clinician. An effective amount of the multispecific molecule may vary according to factors such as the disease state, age, sex, and weight of the individual, and the ability of the antibody to elicit a desired response in the individual. An effective amount is also one in which any toxic or detrimental effect of the antibody is outweighed by the therapeutically beneficial effects. Such benefit includes improving signs or symptoms of cancer. An effective amount of a multispecific molecule of the present invention may be administered in a single dose or in multiple doses. In determining the effective amount for a patient, a number of factors are considered by the attending medical practitioner, including, but not limited to: the patient's size (e.g., weight or mass), body surface area, age, and general health; the specific disease or disorder involved; the degree of, or involvement, or the severity of the disease or disorder; the response of the individual patient; the particular compound administered; the mode of administration; the bioavailability characteristics of the preparation administered; the dose regimen selected; the use of concomitant medication; and other relevant circumstances known to medical practitioners.

### EXAMPLES

### Example 1: Antibody Binding as Determined by Surface Plasmon Resonance

Antibodies binding to ASGR1 in a pH/Ca2+-dependent manner were screened by employing surface plasmon resonance. Antibodies were screened either at pH 7.4 with 2 mM CaCl2 or at pH 6.0 with 2 μM CaCl2 to analyze the binding to ASGR1. K_{D} values were determined by immobilizing receptor on the chip and antibodies as analytes.

The binding of anti-ASGR1 antibodies binding ASGR1 in a pH/Ca2+ dependent or independent manner was assessed by BIAcore. Antibody that binds ASGR1 in a non-catabolic manner comprises a HC amino acid sequence given by SEQ ID NO: 9, and a LC amino acid sequence given by SEQ ID NO: 10. Antibody that binds ASGR1 in a catabolic manner comprises a HC amino acid sequence given by SEQ ID NO: 19, and a LC amino acid sequence given by SEQ ID NO: 20.

Equilibrium binding affinities of the interaction between mouse ASGR1 and anti-ASGR1 antibodies were determined using BIAcore 3000. Mouse ASGR1 protein was obtained from R&D-systems (cat #2755-AS/CF) and immobilized on CM5 chips using amine coupling chemistry to a density of ˜1500 RU. On each CM5 chip a reference flow cell is used which was coupled with coupling buffer only. Antibodies were injected over immobilized ASGR1 at a concentration range of 1000 nM-0.2 nM, with a two-fold serial dilution. To determine pH/Ca-sensitive binding, runs were performed using phosphate buffered saline (PBS) with 0.01% (v/v) Tween20 and 0.05% azide with either pH 7.4/2 mM calcium chloride or pH 6.0/2 μM calcium chloride. The chip was regenerated using 0.15 M NaCl, 0.1 M glycine pH 1.5 buffer between each injection cycles. Equilibrium dissociation constants were determined using 1:1 interaction model using BIAevaluation.

Anti-ASGR1 antibodies exhibiting no differential binding at either pH 7.4/2 mM calcium chloride or pH 6.0/2 μM calcium chloride is termed non-catabolic antibody (non-CAT mAb), whereas antibody exhibiting no detectable binding at pH 6.0/2 μM calcium chloride is termed catabolic antibody (CAT mAb).

These data demonstrate that CAT mAb and non-CAT mAb have 20 nM and 6 nM affinity towards ASGR1, respectively at pH 7.4 and 2 mM CaCl2 (Table 1). Non-CAT mAb exhibited 5.5 nM binding affinity towards ASGR1 at low pH/Ca2+ condition, while non-CAT mAb has no detectable binding at this analyzed condition (Table 1).

TABLE 1
Equilibrium dissociation constants of the interactions
between mouse ASGR1 and antibodies at pH 7.4 with
2 mM CaCl2 or at pH 6.0 with 2 μM CaCl2.

| | muASGR1 | muASGR1 |
|---|---|---|
| | [K_{D}, nM] | [K_{D}, nM] |
| | (pH 7.4, 2 mM | (pH 6, 2 μM |
| Antibody | CaCl₂) | CaCl₂) |
| Non-CAT-WT | 6.0 | 5.5 |
| CAT-WT | 20 | N.D. |

### Example 2: In-Vivo Pharmacokinetics

Pharmacokinetic parameters of antibodies observed in homozygous huFcRn Tg32 mice have high correlation to parameters observed in non-human primates and humans (see e.g. Avery et al., mAbs 2016, 8 (6), 1064-1078). To determine the in vivo pharmacokinetics of anti-ASGR1 antibodies, pharmacokinetic experiments were conducted in 8-12 week old male or female C57BL/6 mice harboring human FcRn gene (homozygous huFcRn Tg32 mice; Jackson laboratory, stock #014565) and in wild type C57BL/6 mice. Mice were administered CAT ASGR1 or non-CAT ASGR1 antibodies intravenously via the lateral tail vein in 150 μl of respective antibody buffer in each animal. Doses ranged from 0.3-30 mg/kg. At the indicated time points, 50 μl of whole blood was collected using SARSTEDT Microvette® serum separator tubes via submandibular vein puncture using a sparse serial sampling scheme (n=3 mice/group/time point). Whole blood was allowed to clot at room temperature for 20 minutes prior to centrifugation at 11,500 rpm for 15 minutes and resulting serum was stored at −70° C. until further analysis. Non-compartmental analysis was performed on the concentration time profiles of the dosed antibodies. AUClast was calculated for each individual animal and the mean is presented in Table 2 along with the standard deviation in the brackets.

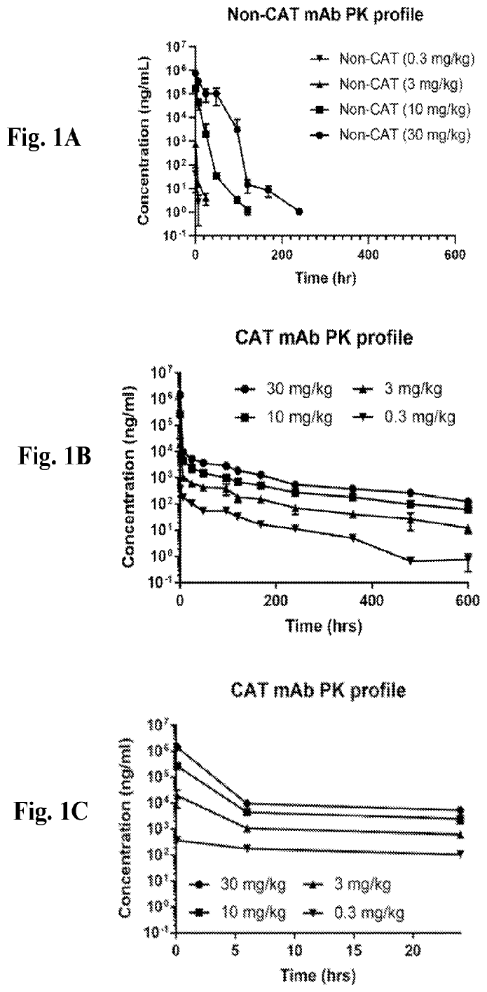

Dosed non-CAT-WT antibody exhibited no dose proportional exposure indicating substantial TMDD (target mediated drug disposition) clearance (FIG. 1A). Rapid decline with no detectable antibody concentration post 24 hours after administration of 0.3 mg/kg and 3 mg/kg non-CAT-WT antibody indicates rapid binding and degradation of bound antibody by ASGR1. At 10 mg/kg dosing, clearance profile is biphasic where rate of clearance marginally decreased post 48 hours after administration. Typical four phases of TMDD mediated clearance were observed for antibody dosed at 30 mg/kg (FIG. 1A). In contrast, CAT antibody displayed modulated clearance profile when compared to non-CAT antibody. At all doses levels, CAT antibody exhibited enhanced rapid decline in concentration within 24 hours post administration (FIG. 1B). Post 24 hours after administration, CAT antibody rate of clearance decreased substantially, suggesting minimal ASGR1 mediated clearance during this period. CAT antibody designed to overcome TMDD was effective at low dose levels (Table 2). At 0.3 mg/kg and 3 mg/kg doses the exposure of CAT antibody is 84.3 and 58-fold excess of non-CAT antibody, respectively. At 10 mg/kg dose, both CAT and non-CAT antibodies have similar exposure (Table 2). But at 30 mg/kg dose, CAT antibody has reduced exposure and is 0.4-fold of non-CAT antibody. Clearance properties of both CAT and non-CAT antibodies indicate moieties targeting ASGR1 could be employed to deplete soluble antigens by crosslinking them to anti-ASGR1 antibodies.

TABLE 2
Serum exposure of dosed antibodies.

| Dose | | | | |
|---|---|---|---|---|
| (mg/kg) | Non-CAT mAb | | CAT mAb | |
| 0.3 | 160 | [98.2] | 13488 | [175] |
| 3 | 2551 | [431.1] | 147905 | [47063.6] |
| 10 | 1057158.6 | [209798.1] | 1136542.3 | [61451] |
| 30 | 12612420.6 | [532286] | 5123802.5 | [158527] |

These data demonstrate that CAT antibodies have faster clearance in-vivo compared to non-CAT antibodies at higher administered doses.

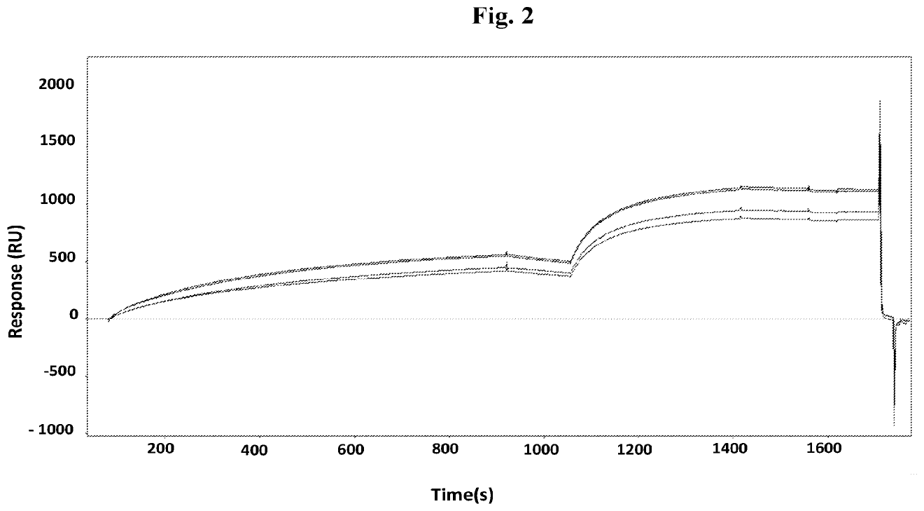

Additional studies demonstrate simultaneous binding of multispecific molecules of the present invention to human IgGs and ASGR1. Catabolic bispecific scFv was immobilized on a CM5 chip, and analytes ASGR1 and IVIg were sequential injected over immobilized bispecific scFv. ASGR1 was injected at 50 nM and 100 nM, and IVIg was injected at 100 nM. Runs were performed in duplicate and both runs are presented in FIG. 2. Binding of IVIg to the complexed ASGR1:bispecific scFv indicate a bispecific scFv of the present invention can simultaneously bind to ASGR1 and IVIg.

### Example 3: Format and Manufacturability of Multispecific Molecules

Based on the in-vivo clearance of catabolic and non-catabolic anti-ASGR1 antibodies, anti-ASGR1 moieties can be utilized as vehicles to deplete soluble antigens. To target autoantibody mediated diseases requires depletion of circulating human Igs (see e.g. Howard et al., Neurology 2019, 92(23). Igs (e.g. IgG) can be cross-linked with ASGR1 to facilitate the clearance of Igs).

Two key design principles were considered while designing the format of the bispecific scFv molecules of the present invention, which cross-link ASGR1 and human IgGs. Firstly, to negate the formation of IgG complex by bispecific scFv molecules of the present invention, which activate immune system resulting in anaphylaxis or other immune mediated side-effects (see e.g. Mayadas et al., Circulation 120 (20), 17 Nov. 2009: 2012-2024), moiety targeting IgG binding shall be monovalent. Second, bispecific scFv molecules of the present invention shall be devoid of human IgG fragments which can lead to self-binding. For example, bispecific scFv molecules of the present invention shall be devoid of Fc fragment if Fc fragment is being targeted to deplete IgGs. Similarly, bispecific scFv molecules of the present invention shall also be devoid of Fc fragment if Fc fragment is being targeted to deplete IgGs.

To generate bispecific scFvs, antibody sequences were reformatted to scFvs with antibody heavy chain and light chain fused by 3×G4S linker and followed by 6×His at the C-terminus. Gene fragments for scFvs were synthesized and cloned into mammalian stable expression, a pTT5 derived vector carrying a puromycin selection marker. Secreted scFv protein was directly captured from the conditioned media by affinity chromatography using Ni Sepharose Excel resin (GE Healthcare Life Sciences). Additional polishing was accomplished using either CHTTM ceramic hydroxyapatite Type I 40 um resin (Bio-Rad) eluted with a linear gradient of sodium phosphate or Source 15S resin (GE Healthcare Life Sciences) eluted with a linear NaCl gradient. The final protein was buffer exchanged by dialysis into the final formulation: 25 mM citrate, 75 mM arginine, 4% sucrose, pH 7.0. Final product quality was confirmed by mass spectrometry (Agilent 1260 Infinity Binary UHPLC/6230 Time-of-Flight Mass Spectrometer), HPLC-SEC (Agilent 1100) and endotoxin testing (Charles River EndoSafe MCS). Bispecific scFvs were chosen for further in-vitro and in-vivo experiments. Bispecific scFv molecule that bind ASGR1 in a non-catabolic manner and Ig in a non-catabolic manner (non-CAT bispecific scFv molecule) comprises an amino acid sequence given by SEQ ID NO: 37. Bispecific scFv molecule that binds ASGR1 in a catabolic manner and Ig in a non-catabolic manner (CAT bispecific scFv molecule) comprises an amino acid sequence given by SEQ ID NO: 46.

### Example 4: Bispecific scFv Molecules Simultaneously Bind to ASGR1 and Human IgGs

To demonstrate simultaneous binding of bispecific scFv molecules of the present invention to human IgGs and ASGR1, ASGR1 was immobilized on SPR chip followed by co-administration of 100 nM bispecific scFv molecule (comprising a binding domain that binds ASGR1 in a catabolic manner and a binding domain that binds IgG in a non-catabolic manner) or anti-ASGR1 antibody (comprising a binding domain that binds ASGR1 in a catabolic manner) of the present invention and 100 nM IVIg (intravenous immunoglobulin). IVIg comprises of pool of immunoglobulins from a large cohort of healthy human volunteers. K_{D} values were determined by immobilizing receptor on the chip and antibodies as analytes.

The binding of anti-ASGR1 antibodies binding ASGR1 in a pH/Ca2+ dependent or independent manner was assessed by BIAcore. Equilibrium binding affinities of the interaction between (i) mouse or human ASGR1 and anti-ASGR1 antibodies; and (ii) mouse or human ASGR1 and bispecific scFv molecules of the present inventions were determined using BIAcore 3000. Mouse and human ASGR1 proteins were obtained from R&D-systems (cat #2755-AS/CF and 4394-AS/CF, respectively) and immobilized on CM5 chips using amine coupling chemistry to a density of ˜1500 RU. On each CM5 chip a reference flow cell is used which was coupled with coupling buffer only. Antibodies were injected over immobilized ASGR1 at a concentration range of 1000 nM-0.2 nM, with a two-fold serial dilution. To determine pH/Ca-sensitive binding, runs were performed using phosphate buffered saline (PBS) with 0.01% (v/v) Tween20 and 0.05% azide with either pH 7.4/2 mM calcium chloride or pH 6.0/2 μM calcium chloride. The chip was regenerated using 0.15 M NaCl, 0.1 M glycine pH 1.5 buffer between each injection cycles. Equilibrium dissociation constants were determined using 1:1 interaction model using BIAevaluation.

These data demonstrate that a bispecific scFv molecule of the present invention exhibited dose dependent binding to immobilized ASGR1, and binding of IVIg to the complexed ASGR1:bispecific scFv molecules of the present invention indicate bispecific scFv molecules of the present invention can simultaneously bind to ASGR1 and IVIg. Anti-huIgG component of bispecific scFv molecules of the present invention binds to IgG1, IgG2 and IgG4 sub-classes of human IgG. Due to conversion from mAb format to scFv, bispecific scFv molecules of the present invention in scFv has a decrease of affinity towards ASGR1 (Table 3). These data demonstrate that in bispecific scFv format, bispecific scFv molecules of the present invention (bispecific scFv) can bind to ASGR1, and human IgGs simultaneously. Binding of one target to bispecific scFv does not inhibit the binding to the other target.

TABLE 3
Equilibrium dissociation constants of the interactions between
mouse ASGR1 and anti-ASGR1 antibodies or bispecific scFv molecules
of the present invention at pH 7.4 with 2 mM CaCl₂.

| | pH 7.4 with |
|---|---|
| Molecule | 2 mM Ca²⁺ [nM] |
| CAT-bispecific scFv | 78.3 |
| molecule:muASGR1 | |
| CAT- bispecific scFv | 23 |
| molecule:huASGR1 | |
| CAT anti-ASGR1:muASGR1 | 12.1 |
| CAT anti-ASGR1:huASGR1 | 4 |

### Example 5: In Vivo Clearance

Administering IVIg in animals mimics clinical scenario of circulatory IgGs in-vivo (Schwab I and Nimmerjahn F, Nat. Rev. 2013, 13, 176-189). Moieties blocking FcRn mediated IgG recycling such as anti-FcRn antibodies or ABDEG require approximately four days to deplete human IgGs administered in mice by about 70-80%, and that ABDEG takes 2-3 weeks in humans to clear ˜50-70% IgGs (Vaccaro C et al., Nat. Biotechnol. 23, 1283-1288 (2005); Getman K E & Balthasar J P, J. Pharm. Sci. 94, 718-729 (2005); Mezo A R et al., Proc. Natl. Acad. Sci. 2008, 105, 2337-2342; Peter U et al., J. Clin. Invest. 2018, 128(10), 4372-4386; and James F H et al., Neurology, 2019, 92(23)).

To determine the clearance of exogenously administered IVIg in mice and in non-human primates, clearance was determined in mice and cynomolgus monkeys exogenously administered with IVIg and bispecific scFv molecule of the present invention binding ASGR1 with a pH and calcium sensitivity.

To analyze the clearance of human IgGs, mice were administered with IVIG (Sigma, cat #56834) intravenously, and post 72 hours mice were administered either with CAT bispecific scFv molecule (that binds ASGR1 in a catabolic manner and Ig in a non-catabolic manner) of the present invention (1.67 μM) or PBS. At indicated time points, 50 μl of whole blood was collected using SARSTEDT Microvette® serum separator tubes via submandibular vein puncture using a sparse serial sampling scheme (n=3 mice/group/time point). Whole blood was allowed to clot at room temperature for 20 minutes prior to centrifugation at 11,500 rpm for 15 minutes and resulting serum was stored at −80° C. until further analysis.

For cynomolgus monkey analysis, female drug naïve cynomolgus monkeys were administered an intravenous dose of bispecific scFv molecules of the present invention via a saphenous vein and blood was collected via a femoral vein into tubes containing no anticoagulant (serum separator tubes) at indicated time points. Blood was allowed to clot at ambient temperature prior to centrifugation to obtain serum. Centrifugation began within one hour of collection. Serum was placed in polypropylene tubes and maintained on dry ice prior to storage at −80° C.

For quantification of huIVIG, mouse anti-human IgG, F(ab′)2 specific antibody (Jackson ImmunoResearch Labs, cat #209-005-097) was used as a capture and detection reagent in an ELISA based assay.

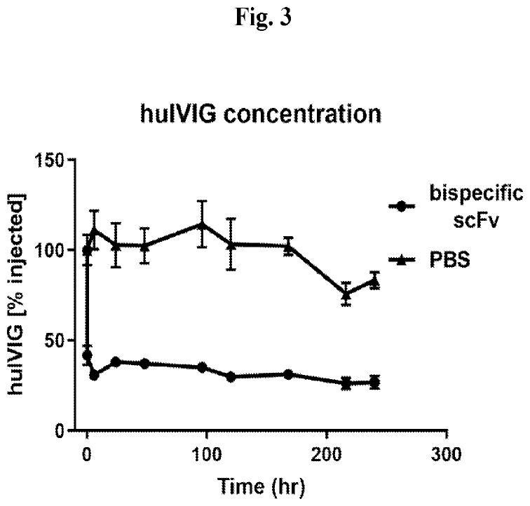

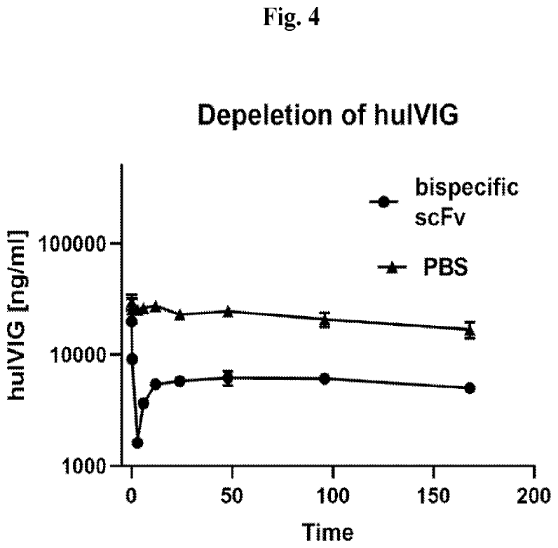

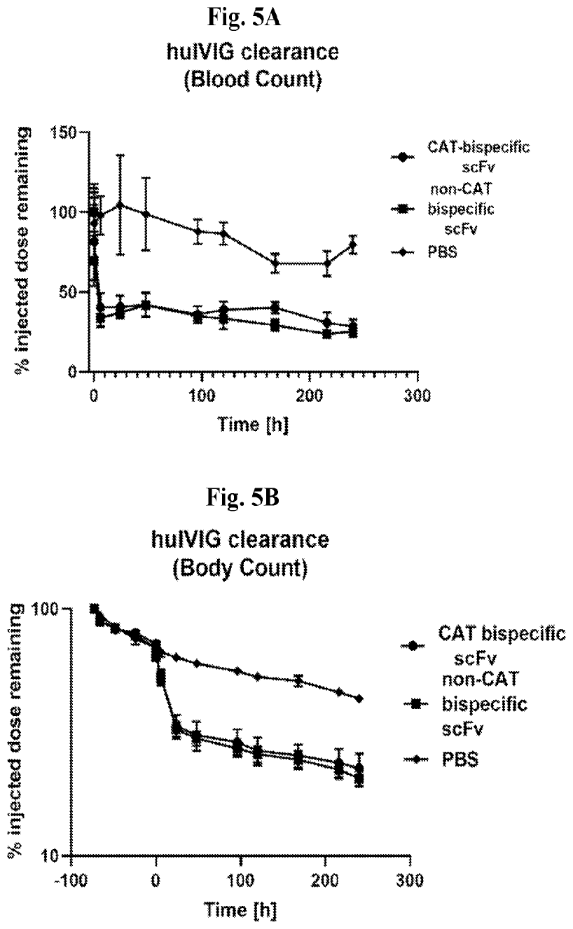

These data demonstrate that human IgG concentration in mice decreased by ˜70% within three hours post administration of a CAT bispecific scFv molecule of the present invention (FIG. 3). Similar enhanced clearance effect of bispecific scFv molecules of the present invention was observed in cynomolgus monkeys, in which bispecific scFv molecule of the present invention deplete human IgGs by 72% within post 12 hours of administration of bispecific scFv molecules of the present invention (FIG. 4).

### Example 6: CAT and Non-CAT Bispecific scFv Molecule Generation

As shown in FIG. 1, anti-ASGR1 antibody binding to the receptor in a catabolic manner had faster in-vivo clearance in comparison to non-catabolic anti-ASGR1 antibody. This rapid clearance is indicative of faster accumulation of catabolic anti-ASGR1 to lysosomes. To analyze if rapid clearance of catabolic anti-ASGR1 moiety will lead to faster clearance of human IgGs if bispecific scFv molecules of the present invention binds to ASGR1 in a catabolic manner (and Ig in a non-catabolic manner), bispecific scFv molecules of the present invention binding to ASGR1 in catabolic and non-catabolic manner were tested in vivo. Bispecific scFv molecule that binds ASGR1 in a non-catabolic manner comprises an amino acid sequence given by SEQ ID NO: 37. Bispecific scFv molecule that binds ASGR1 in a catabolic manner comprises an amino acid sequence given by SEQ ID NO: 46.

Human IgG (IVIg) were labeled with Na125I (Perkin Elmer, Cat #NEZ033L), and radioactive-based pharmacokinetics was assessed. Mice were injected with 1.67 uM human IVIg via tail vein, and the radioactivity of animals was immediately measured in dose calibrator (Capintec, cat. #CRC-15R) for initial (T=0) whole body activity. Mice were then injected with 1.67 μM bispecific scFv molecule of the present invention via tail vein. At the indicated times the animal was read for whole body activity and then a blood sample was taken by a prick to the tail vein and collected in a capillary tube. All capillary tubes were weighed before collection and then after to determine exact blood weight/volume collected. Radioactivity of the serum samples were analyzed by gamma counter.

As shown in FIG. 5, bispecific scFv molecules of the present inventions binding ASGR1 in either a catabolic or a non-catabolic fashion exhibited similar efficacy in depleting the serum IgGs and catabolizing them in vivo, suggesting that pH/Ca2+-dependent binding to ASGR1 did not have major influence on bispecific scFv molecules of the present inventions for clearing exogenously administered human IgGs in mice.

### Example 7: Bispecific scFv Molecule Induced IgG Liver Catabolization

ASGR1 is primarily expressed in hepatocytes both on the cell membrane and in the cytoplasm, specifically on the limiting membrane of the endosomes. Ligands targeting ASGR1 anticipated to be accumulated and catabolized in the liver.

To analyze the catabolization of exogenously administered IVIg in mice post administration of targeting molecule, IVIg is labelled with non-residualizing and residualizing radioactive labels I-125 and In-111, respectively. Iodine, a non-residualizing dye, is secreted from the cell upon cleavage from IgG when IgG is degraded, after which it undergoes renal clearance. Indium, however, is a residualizing dye and hence it remains in the cell even after being cleaved from IgG. Therefore, similar levels of I-125 and In-111 indicates a lack of catabolism, whereas different levels indicate IgG is being catabolized in the liver.

Radiolabeled IVIg was administered to mice via tail vein injection, and the radioactivity of animals was measured in dose calibrator (Capintec, cat. #CRC-15R) for initial (T=0) whole body activity. After 72 hours, animals were administered with either CAT bispecific scFv molecule of the present invention (binding ASGR1 in catabolic manner and Ig in a non-catabolic manner) or PBS. At indicated time points the animals were read for whole body activity and then a blood sample was taken by a prick to the tail vein and collected in a capillary tube. All capillary tubes were weighed before collection and then after to determine exact blood weight/volume collected. Radioactivity of the serum samples are analyzed by gamma counter. The animals were perfused followed by organs being harvested, weighed, and measured for radioactivity.

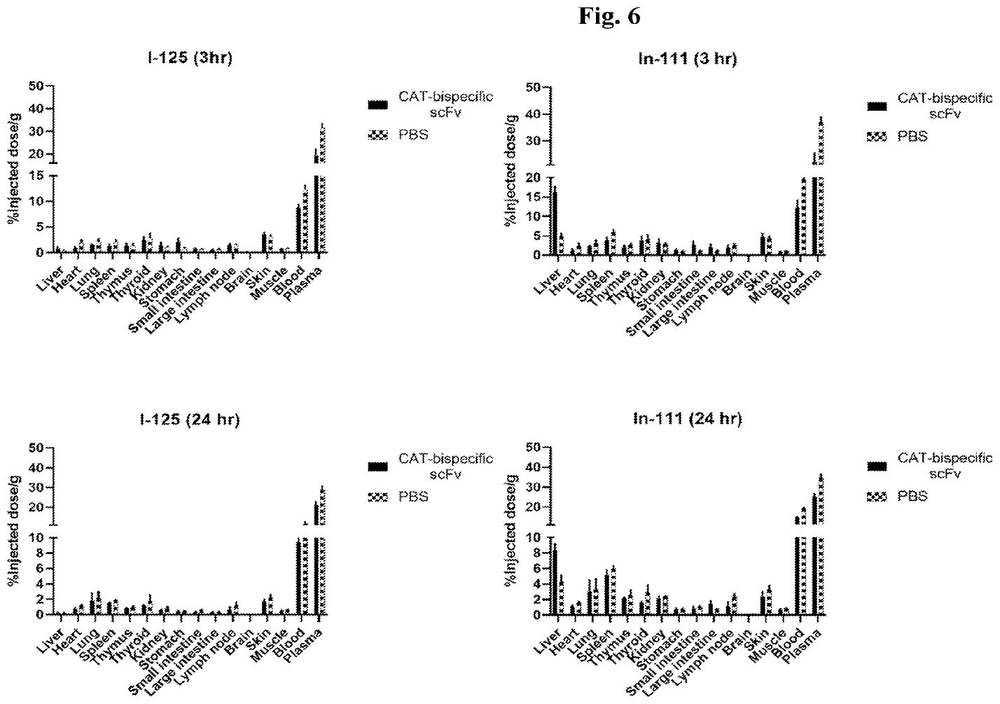

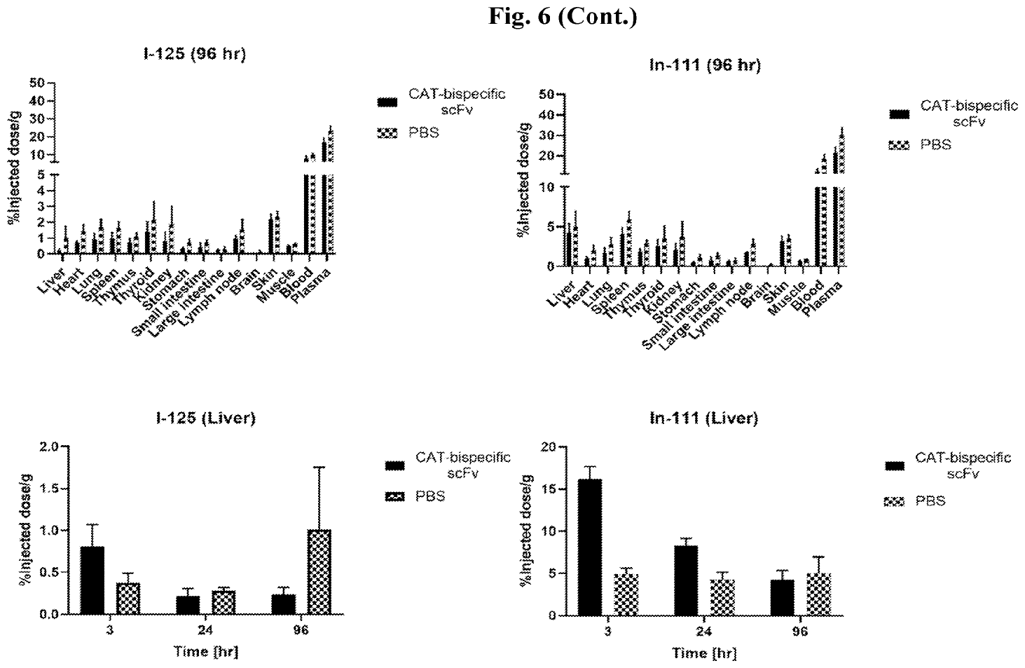

These data, shown in FIG. 6, demonstrate that by three hours after administration of targeting molecule, the majority of the circulating human IgGs localized to liver as indicated by the radioactivity of residualizing label In-111 compared to the PBS control group. The accumulated human IgGs catabolized rapidly by hepatic lysosomes as the radioactivity signal of non-residualizing label I-125 is ˜1% ID/g at all the measured time points. Bringing antigens, such as Ig, to the liver to be cleared from circulation is thought to result in reduced toxicity compared to clearing antigens via a different mechanism.

### Example 8: Catabolic IgG Molecules

Molecules that bind to ASGR1 in a non-catabolic manner and bind to IgG in a catabolic manner were generated. This binding characteristic to ASGR1 and IgG would enable continuous recycling of said molecules by binding to ASGR1, while dissociating IgG in the endosomal compartment. Mouse IgG2a was chosen as an antigen to enable to test the generated molecules in in-vivo disease models which express autoantibodies of IgG2a subclass.

Rabbits were immunized with mouse IgG2a, and B-cells spleens of immunized animals were harvested. Harvested cells were sorted through FACS based multiplex assay by analyzing binding to mouse IgG2a, IgG2b, IgG2c, IgG1 and irreverent antigen. Clones binding to mouse IgG2a specifically were identified and further screened for their catabolic binding by analyzing the binding at pH 6.0 with 2 μM CaCl2 and at pH 7.4 with 2 mM CaCl2. Antibody heavy and light chain sequences were extracted for the binders exhibiting desired catabolic binding properties, binders were converted into Fab-scFc format (anti-mIgG2a), and analyzed for binding characteristics to mouse IgG2a for their catabolic binding affinities through BIAcore and Octet based assays.

For Octet based assays, streptavidin biosensors were loaded with avidin coupled mouse IgG2a and association and dissociation of binders were conducted with above described acidic and neutral pH conditions. The biosensor was regenerated with 10 mM glycine buffer at pH 1.5.

Similarly, for BIAcore based assays, anti-mouse IgG (H+L) cross adsorbed was amine coupled to a CM5 sensor chip and mouse IgG2a was captured as ligand, while generated binders were injected as analytes either at neutral pH with 2 mM CaCl2 or at acidic pH with 2 μM CaCl2. Analytes were injected in a serial dilution of 3-fold, with highest concentration ranging from 900 nM. The CM5 chip was regenerated with 10 mM glycine buffer at pH 1.5. The analysis of binding was performed in BIAevaluation using 1:1 langmuir fit.

Octet data for molecule 099 (comprising a HC comprising SEQ ID NO: 53, and a LC comprising SEQ ID NO: 54) is shown in Table 4. Molecule 099 exhibited tight binding to mouse IgG2a at neutral pH with 2 mM CaCl2, while exhibiting reducing affinity (˜3-fold) towards mouse IgG2a at acidic pH with 2 μM CaCl2). The weaker the affinity towards the antigen at acidic pH greater the efficiency of release of antigen in the endosomes.

Molecule 099 was further engineered by introducing histidine residues in the CDR regions to enhance the pH-dependent binding. Two resulting clones (465, comprising a HC comprising SEQ ID NO: 55, and a LC comprising SEQ ID NO: 56; and 463 comprising a HC comprising SEQ ID NO: 57, and a LC comprising SEQ ID NO: 58) demonstrated further reduced binding to mouse IgG2a at pH 6.0 with 2 μM CaCl2, while retaining tight binding at neutral pH with 2 mM CaCl2 (Table 5). Resulting sensograms (BIAcore data) demonstrated similar catabolic binding characteristics to mouse IgG2a as determined via Octet assays.

TABLE 4
Octet-based binding characterization of generated molecules to mouse
IgG2a at neutral pH with 2 mM CaCl2 and at acidic pH with 2 μM CaCl2.

| | KD | ka | kdiss | KD | ka | kdiss | |
|---|---|---|---|---|---|---|---|
| | (M) | (1/Ms) | (1/s) | (M) | (1/Ms) | (1/s) | |
| | (pH 7.4, | (pH 7.4, | (pH 7.4, | (pH 6, | (pH 6, | (pH 6, | pH |
| | 2 mM | 2 mM | 2 mM | 2 μM | 2 μM | 2 μM | 6/pH |
| Molecule | CaCl2) | CaCl2) | CaCl2) | CaCl2) | CaCl2) | CaCl2) | 7.4 |
| 099 | 3.0E−09 | 8.1E+04 | 2.4E−04 | 9.3E−09 | 7.4E+04 | 6.8E−04 | 2.8 |

TABLE 5
Octet-based binding characterization of generated molecules to mouse
IgG2a at neutral pH with 2 mM CaCl2 and at acidic pH with 2 μM CaCl2.

| | KD | ka | kdiss | KD | ka | kdiss | |
|---|---|---|---|---|---|---|---|
| | (M) | (1/Ms) | (1/s) | (M) | (1/Ms) | (1/s) | |
| | (pH 7.4, | (pH 7.4, | (pH 7.4, | (pH 6, | (pH 6, | (pH 6, | pH |
| | 2 mM | 2 mM | 2 mM | 2 μM | 2 μM | 2 μM | 6/pH |
| Molecule | CaCl2) | CaCl2) | CaCl2) | CaCl2) | CaCl2) | CaCl2) | 7.4 |
| 465 | 1.0E−08 | 5.4E+04 | 5.4E−04 | 2.6E−04 | 8.7E+01 | 2.2E−02 | 41.1 |
| 463 | 5.0E−09 | 4.0E+04 | 2.0E−04 | 5.8E−07 | 1.7E+04 | 1.0E−02 | 50.0 |

These data demonstrate the tested molecules bind to IgG in a catabolic manner. They have reduced binding to mouse IgG2a at pH 6.0 with 2 μM CaCl2, while retaining tight binding at neutral pH with 2 mM CaCl2.

The binders were converted into scFv-scFv format by converting them to scFv and fusing them with anti-mouse/human ASGR1 scFv (for example, SEQ ID NO: 75). Additional cysteine(s) were introduced to improve stability of the bispecific scFv molecules. These bispecific scFvs were analyzed for binding to mouse/human ASGR1 and mouse IgG2a through flow-based assay and were determined to retain binding to ASGR1 and IgG2a when converted from Fab to scFv format at neutral pH.

SEQUENCES

Non-Catabolic Anti-ASGR1 Antibody HCDR1 (SEQ ID NO: 1)
DYNMA
Non-Catabolic Anti-ASGR1 Antibody HCDR2 (SEQ ID NO: 2)
TIIYDGGSTYYRHSVKG
Non-Catabolic Anti-ASGR1 Antibody HCDR3 (SEQ ID NO: 3)
QTYFGSRDYFDY
Non-Catabolic Anti-ASGR1 Antibody LCDR1 (SEQ ID NO: 4)
LTSEDIYNNLA
Non-Catabolic Anti-ASGR1 Antibody LCDR2 (SEQ ID NO: 5)
YASNFQD
Non-Catabolic Anti-ASGR1 Antibody LCDR3 (SEQ ID NO: 6)
LQDSEYPP
Non-Catabolic Anti-ASGR1 Antibody HCVR (SEQ ID NO: 7)
EVQLVESGGGLVQPGRSLKLSCAASGFTFSDYNMAWVRQAPKKGLEWVATIIYDGGST
YYRHSVKGRFTISRDNAKSTHSLQMDSLRSEDTATYYCARQTYFGSRDYFDYWGQGVM
VTVSS
Non-Catabolic Anti-ASGR1 Antibody LCVR (SEQ ID NO: 8)
DIQMTQSPTSLSASLGETVSIECLTSEDIYNNLAWYQQKPGKSPQLLISYASNFQDGVPSR
FSGSGSGTQYSLKINSLESEDAATYFCLQDSEYPPTFGGGTKLELKR
Non-Catabolic Anti-ASGR1 Antibody HC (SEQ ID NO: 9)
EVQLVESGGGLVQPGRSLKLSCAASGFTFSDYNMAWVRQAPKKGLEWVATIIYDGGST
YYRHSVKGRFTISRDNAKSTHSLQMDSLRSEDTATYYCARQTYFGSRDYFDYWGQGVM
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAKTKP
CEEQYGSTYRCVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK (SEQ ID NO: 10)
Non-Catabolic Anti-ASGR1 Antibody LC
DIQMTQSPTSLSASLGETVSIECLTSEDIYNNLAWYQQKPGKSPQLLISYASNFQDGVPSR
FSGSGSGTQYSLKINSLESEDAATYFCLQDSEYPPTFGGGTKLELKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
Catabolic Anti-ASGR1 Antibody HCDR1 (SEQ ID NO: 11)
SYGMH
Catabolic Anti-ASGR1 Antibody HCDR2 (SEQ ID NO: 12)
VIWYDGSNKYYADSVKG
Catabolic Anti-ASGR1 Antibody HCDR3 (SEQ ID NO: 13)
DSSPYGMDV
Catabolic Anti-ASGR1 Antibody LCDR1 (SEQ ID NO: 14)
RASQGISSWLA
Catabolic Anti-ASGR1 Antibody LCDR2 (SEQ ID NO: 15)
GASSLQS
Catabolic Anti-ASGR1 Antibody LCDR3 (SEQ ID NO: 16)
QQSDSFPRT
Catabolic Anti-ASGR1 Antibody HCVR (SEQ ID NO: 17)
QVQLVESGGGVVQPGRSLRLSCAASGFTFSSYGMHWVRQAPGKGLEWVAVIWYDGSN
KYYADSVKGRFTISRDNSKNTLYLQMNSLRAEDTAVYYCARDSSPYGMDVWGQGTTV
TVSS
Catabolic Anti-ASGR1 Antibody LCVR (SEQ ID NO: 18)
DIQMTQSPSSVSASVGDRVTITCRASQGISSWLAWYQQKPGKAPKLLIYGASSLQSGVPS
RFSASGSGTDFTLTISSLQPEDFATYYCQQSDSFPRTFGQGTKVEIKR
Catabolic Anti-ASGR1 Antibody HC (SEQ ID NO: 19)
QVQL VESGGGVVQPGRSLRLSCAASGFTFSSYGMHWVRQAPGKGLEWVAVIWYDGSN
KYYADSVKGRFTISRDNSKNTLYLQMNSLRAEDTAVYYCARDSSPYGMDVWGQGTTV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPCVKFNWYVDGVEVHNAKTKPC
EEQYGSTYRCVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
Catabolic Anti-ASGR1 Antibody LC (SEQ ID NO: 20)
DIQMTQSPSSVSASVGDRVTITCRASQGISSWLAWYQQKPGKAPKLLIYGASSLQSGVPS
RFSASGSGTDFTLTISSLQPEDFATYYCQQSDSFPRTFGQGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
Anti-IgG HCDR1 (SEQ ID NO: 21)
DTYIH
Anti-IgG HCDR2 (SEQ ID NO: 22)
RIDPANGNTKYDPKFQD
Anti-IgG HCDR3 (SEQ ID NO: 23)
NYGSNYDPMDY
Anti-IgG LCDR1 (SEQ ID NO: 24)
RASQNIDTNIH
Anti-IgG LCDR2 (SEQ ID NO: 25)
YASESIS
Anti-IgG LCDR3 (SEQ ID NO: 26)
QQSDTWPWT
Non-Catabolic anti-ASGR1 HCDR1 (SEQ ID NO: 27)
DYSVH
Non-Catabolic anti-ASGR1 HCDR2 (SEQ ID NO: 28)
IMWTGGSTAYNSALKS
Non-Catabolic anti-ASGR1 HCDR3 (SEQ ID NO: 29)
DGDYGPDY
Non-Catabolic anti-ASGR1 LCDR1 (SEQ ID NO: 30)
QASQDIGNWLS
Non-Catabolic anti-ASGR1 LCDR2 (SEQ ID NO: 31)
GATSLAD
Non-Catabolic anti-ASGR1 LCDR3 (SEQ ID NO: 32)
LQAYSAPPWT
Anti- IgG HCVR (SEQ ID NO: 33)
ELQLQQSGAELVRPGASVKLSCTTSGFNVKDTYIHWVRQRPEQGLEWIGRIDPANGNTK
YDPKFQDRATITTDTSSITAYLQLSSLTSEDTAVYYCARNYGSNYDPMDYWGQGTSLTV
SS
Anti- IgG LCVR (SEQ ID NO: 34)
DILLTQSPAILSVSPGER VSFSCRASQNIDTNIHWYQRRTNDSPRLLIKYASESISGIPSRFS
GSGSGTDFTLSINSVESEDIADYYCQQSDTWPWTFGGGTKLEIKR
Non-Catabolic anti-ASGR1 HCVR (SEQ ID NO: 35)
EVQLKESGPGLVQPSQTLSLTCTVSGFSLTDYSVHWVRQSPGKGLEWMGIMWTGGSTA
YNSALKSRLSISRDTSKSQVFLKMNSLQTEDTAIYYCTRDGDYGPDYWGQGVMVTVSS
Non-Catabolic anti-ASGR1 LCVR (SEQ ID NO: 36)
DIQMTQSPASLSASLEEIVTITCQASQDIGNWLSWYQQKPGKSPQLLIYGATSLADGVPSR
FSGSRSGTQYSLKISRLQVEDIGIYYCLQAYSAPPWTFGGGTKLELKR
Non-Catabolic bispecific scFv (SEQ ID NO: 37)
ELQLQQSGAELVRPGASVKLSCTTSGFNVKDTYIHWVRQRPEQGLEWIGRIDPANGNTK
YDPKFQDRATITTDTSSITAYLQLSSLTSEDTAVYYCARNYGSNYDPMDYWGQGTSLTV
SSGGGGSGGGGSGGGGSDILLTQSPAILSVSPGERVSFSCRASQNIDTNIHWYQRRTNDSP
RLLIKYASESISGIPSRFSGSGSGTDFTLSINSVESEDIADYYCQQSDTWPWTFGGGTKLEI
KRSGGGGSEVQLKESGPGLVQPSQTLSLTCTVSGFSLTDYSVHWVRQSPGKGLEWMGI
MWTGGSTAYNSALKSRLSISRDTSKSQVFLKMNSLQTEDTAIYYCTRDGDYGPDYWGQ
GVMVTVSSGGGGSGGGGSGGGGSDIQMTQSPASLSASLEEIVTITCQASQDIGNWLSWY
QQKPGKSPQLLIYGATSLADGVPSRFSGSRSGTQYSLKISRLQVEDIGIYYCLQAYSAPPW
TFGGGTKLELKRHHHHHH
Catabolic anti-ASGR1 HCDR1 (SEQ ID NO: 38)
SYGMH
Catabolic anti-ASGR1 HCDR2 (SEQ ID NO: 39)
VIWYDGSNKYYADSVKG
Catabolic anti-ASGR1 HCDR3 (SEQ ID NO: 40)
DSSPYGMDV
Catabolic anti-ASGR1 LCDR1 (SEQ ID NO: 41)
RASQGISSWLA
Catabolic anti-ASGR1 LCDR2 (SEQ ID NO: 42)
GASSLQS
Catabolic anti-ASGR1 LCDR3 (SEQ ID NO: 43)
QQSDSFPRT
Catabolic anti-ASGR1 HCVR (SEQ ID NO: 44)
QVQLVESGGGVVQPGRSLRLSCAASGFTFSSYGMHWVRQAPGKGLEWVAVIWYDGSN
KYYADSVKGRFTISRDNSKNTLYLQMNSLRAEDTAVYYCARDSSPYGMDVWGQGTTV
TVSS
Catabolic anti-ASGR1 LCVR (SEQ ID NO: 45)
DIQMTQSPSSVSASVGDRVTITCRASQGISSWLAWYQQKPGKAPKLLIYGASSLQSGVPS
RFSASGSGTDFTLTISSLQPEDFATYYCQQSDSFPRTFGQGTKVEIKR
Catabolic bispecific scFv (SEQ ID NO: 46)
ELQLQQSGAELVRPGASVKLSCTTSGFNVKDTYIHWVRQRPEQGLEWIGRIDPANGNTK
YDPKFQDRATITTDTSSITAYLQLSSLTSEDTAVYYCARNYGSNYDPMDYWGQGTSLTV
SSGGGGSGGGGSGGGGSDILLTQSPAILSVSPGERVSFSCRASQNIDTNIHWYQRRTNDSP
RLLIKYASESISGIPSRFSGSGSGTDFTLSINSVESEDIADYYCQQSDTWPWTFGGGTKLEI
KRSGGGGSQVQLVESGGGVVQPGRSLRLSCAASGFTFSSYGMHWVRQAPGKGLEWVA
VIWYDGSNKYYADSVKGRFTISRDNSKNTLYLQMNSLRAEDTAVYYCARDSSPYGMDV
WGQGTTVTVSSGGGGSGGGGSGGGGSDIQMTQSPSSVSASVGDRVTITCRASQGISSWL
AWYQQKPGKAPKLLIYGASSLQSGVPSRFSASGSGTDFTLTISSLQPEDFATYYCQQSDSF
PRTFGQGTKVEIKRHHHHHH
Leader sequence (SEQ ID NO: 47)
MDMRVPAQLLGLLLLWLRGARC
Nucleic acid sequence encoding the leader sequence of SEQ ID NO: 47 (SEQ ID
NO: 48)
ATGGACATGAGAGTGCCTGCACAGCTGCTGGGCCTGCTGCTGCTGTGGCTGAGAGGC
GCCAGATGC
Leader sequence (SEQ ID NO: 49)
MAWALLLLTLLTQGTGSWA
Nucleic acid sequence encoding the leader sequence of SEQ ID NO: 49 (SEQ ID
NO: 50)
ATGGCCTGGGCTCTGCTGCTCCTCACCCTCCTCACTCAGGGCACAGGGTCCTGGGCC
Anti-Ig HC (SEQ ID NO: 51)
ELQLQQSGAELVRPGASVKLSCTTSGFNVKDTYIHWVRQRPEQGLEWIGRIDPANGNTK
YDPKFQDRATITTDTSSITAYLQLSSLTSEDTAVYYCARNYGSNYDPMDYWGQGTSLTV
SSAKTTPPSVYPLAPGSAAQTNSMVTLGCLVKGYFPEPVTVTWNSGSLSSGVHTFPAVL
QSDLYTLSSSVTVPSSTWPSETVTCNVAHPASSTKVDKKIVPRDCGCKPCICTVPEVSSVF
IFPPKPKDVLTITLTPKVTCVVVDISKDDPEVQFSWFVDDVEVHTAQTQPREEQFNSTFRS
VSELPIMHQDWLNGKEFKCRVNSAAFPAPIEKTISKTKGRPKAPQVYTIPPPKEQMAKDK
VSLTCMITDFFPEDITVEWQWNGQPAENYKNTQPIMDTDGSYFVYSKLNVQKSNWEAG
NTFTCSVLHEGLHNHHTEKSLSHSPGK
Anti-Ig LC (SEQ ID NO: 52)
DILLTQSPAILSVSPGERVSFSCRASQNIDTNIHWYQRRINDSPRLLIKYASESISGIPSRFS
GSGSGTDFTLSINSVESEDIADYYCQQSDTWPWTFGGGTKLEIKRADAAPTVSIFPPSSEQ
LTSGGASVVCFLNNFYPKDINVKWKIDGSERQNGVLNSWTDQDSKDSTYSMSSTLTLTK
DEYERHNSYTCEATHKTSTSPIVKSFNRNEC
Catabolic anti-mIgG2a 099 Fab-scFc HC (SEQ ID NO: 53)
EQLEESGGDLVKPGASLTLTCTASGFSFTSDYYMCWVRQAPGKGLEWIACIGAGDIHTT
YYANWAKGRFTISKTSSTTVTLQMTTLTAADTATYFCARDTYNIGGYTGDFDLWGPGT
LVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGKGGGGSGGGGSGGGGSGGGG
SGGGGSGGGGSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCL VKGFYPSDIA VEWESNG
QPENNYKTTPPVLDSDGSFFLYSKLTVDKSRWQQGNVFSCSVMHEALHNHYTQKSLSLS
PGK
Catabolic anti-mIgG2a 099 Fab-scFc LC (SEQ ID NO: 54)
ALVMTQPPASVSAAVGGTVTINCQASESISTWLAWYQQKPGQPPKLLIYYASTLASGVP
SRFKGSGSGTQFTLTISGVECDDAATYYCAGHKSYSSDDFAFGGGTEVVVKGTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Catabolic anti-mIgG2a 465 Fab-scFc HC (SEQ ID NO: 55)
EQLEESGGDLVKPGASLTLTCTASGFSFTSDYYMCWVRQAPGKGLEWIACHGAGDIHTT
YYANWAKGRFTISKTSSTTVTLQMTTLTAADTATYFCARDTYNIGGYTGDFDLWGPGT
LVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGKGGGGSGGGGSGGGGSGGGG
SGGGGSGGGGSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSLSLS
PGK
Catabolic anti-mIgG2a 465 Fab-scFc LC (SEQ ID NO: 56)
ALVMTQPPASVSAAVGGTVTINCQASESISTWLAWYQQKPGQPPKLLIYYASTLASGVP
SRFKGSGSGTQFTLTISGVECDDAATYYCAGHKSYSSDDFAFGGGTEVVVKGTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Catabolic anti-mIgG2a 463 Fab-scFc HC (SEQ ID NO: 57)
EQLEESGGDLVKPGASLTLTCTASGFSFTSDYYHCWVRQAPGKGLEWIACIGAGDIHTT
YYANWAKGRFTISKTSSTTVTLQMTTLTAADTATYFCARDTYNIGGYTGDFDLWGPGT
LVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGKGGGGSGGGGSGGGGSGGGG
SGGGGSGGGGSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSLSLS
PGK
Catabolic anti-mIgG2a 463 Fab-scFc LC (SEQ ID NO: 58)
ALVMTQPPASVSAAVGGTVTINCQASESISTWLAWYQQKPGQPPKLLIYYASTLASGVP
SRFKGSGSGTQFTLTISGVECDDAATYYCAGHKSYSSDDFAFGGGTEVVVKGTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Catabolic anti-mIgG2a 099 HCDR1 (SEQ ID NO: 59)
SDYYMC
Catabolic anti-mIgG2a 099 HCDR2 (SEQ ID NO: 60)
CIGAGDIHTTYYANWAKG
Catabolic anti-mIgG2a 099 HCDR3 (SEQ ID NO: 61)
DTYNIGGYTGDFDL
Catabolic anti-mIgG2a 099, 465, and 463 LCDR1 (SEQ ID NO: 62)
QASESISTWLA
Catabolic anti-mIgG2a 099, 465, and 463 LCDR2 (SEQ ID NO: 63)
YASTLAS
Catabolic anti-mIgG2a 099, 465, and 463 LCDR3 (SEQ ID NO: 64)
AGHKSYSSDDFA
Catabolic anti-mIgG2a 465 HCDR1 (SEQ ID NO: 65)
SDYYMC
Catabolic anti-mIgG2a 465 HCDR2 (SEQ ID NO: 66)
CHGAGDIHTTYYANWAKG
Catabolic anti-mIgG2a 465 HCDR3 (SEQ ID NO: 67)
DTYNIGGYTGDFDL
Catabolic anti-mIgG2a 463 HCDR1 (SEQ ID NO: 68)
SDYYHC
Catabolic anti-mIgG2a 463 HCDR2 (SEQ ID NO: 69)
CIGAGDIHTTYYANWAKG
Catabolic anti-mIgG2a 463 HCDR3 (SEQ ID NO: 70)
DTYNIGGYTGDFDL
Catabolic anti-mIgG2a 099 HCVR (SEQ ID NO: 71)
EQLEESGGDLVKPGASLTLTCTASGFSFTSDYYMCWVRQAPGKGLEWIACIGAGDIHTT
YYANWAKGRFTISKTSSTTVTLQMTTLTAADTATYFCARDTYNIGGYTGDFDLWGPGT
LVTVSS
Catabolic anti-mIgG2a 099, 463, and 465 LCVR (SEQ ID NO: 72)
ALVMTQPPASVSAAVGGTVTINCQASESISTWLAWYQQKPGQPPKLLIYYASTLASGVP
SRFKGSGSGTQFTLTISGVECDDAATYYCAGHKSYSSDDFAFGGGTEVVVKG
Catabolic anti-mIgG2a 465 HCVR (SEQ ID NO: 73)
EQLEESGGDLVKPGASLTLTCTASGFSFTSDYYMCWVRQAPGKGLEWIACHGAGDIHTT
YYANWAKGRFTISKTSSTTVTLQMTTLTAADTATYFCARDTYNIGGYTGDFDLWGPGT
LVTVSS
Catabolic anti-mIgG2a 463 HCVR (SEQ ID NO: 74)
EQLEESGGDLVKPGASLTLTCTASGFSFTSDYYHCWVRQAPGKGLEWIACIGAGDIHTT
YYANWAKGRFTISKTSSTTVTLQMTTLTAADTATYFCARDTYNIGGYTGDFDLWGPGT
LVTVSS
Catabolic anti-mIgG2a Non-Catabolic ASGR1 bispecific scFv (SEQ ID NO: 75)
EQLEESGGDLVKPGASLTLTCTASGFSFTSDYYMCWVRQAPGKGLEWIACIGAGDIHTT
YYANWAKGRFTISKTSSTTVTLQMTTLTAADTATYFCARDTYNIGGYTGDFDLWGPGT
LVTVSSSGGGGSGGGGSGGGGSALVMTQPPASVSAAVGGTVTINCQASESISTWLAWY
QQKPGQPPKLLIYYASTLASGVPSRFKGSGSGTQFTLTISGVECDDAATYYCAGHKSYSS
DDFAFGGGTEVVVKGSGGGGSEVQLKESGPGLVQPSQTLSLTCTVSGFSLTDYSVHWVR
QSPGKGLEWMGIMWTGGSTAYNSALKSRLSISRDTSKSQVFLKMNSLQTEDTAIYYCTR
DGDYGPDYWGQGVMVTVSSGGGGSGGGGSGGGGSDIQMTQSPASLSASLEEIVTITCQ
ASQDIGNWLSWYQQKPGKSPQLLIYGATSLADGVPSRFSGSRSGTQYSLKISRLQVEDIGI
YYCLQAYSAPPWTFGGGTKLELKRHHHHHH
(Gly₃Ser)₃ (SEQ ID NO: 76)
GGGSGGGSGGGS
(Gly₄Ser)₃ (SEQ ID NO: 77)
GGGGSGGGGSGGGGS
(Gly₃Ser)₄ (SEQ ID NO: 78)
GGGSGGGSGGGSGGGS
(Gly₄Ser)₄ (SEQ ID NO: 79)
GGGGSGGGGSGGGGSGGGGS
(Gly₃Ser)₅ (SEQ ID NO: 80)
GGGSGGGSGGGSGGGSGGGS
(Gly₄Ser)₅ (SEQ ID NO: 81)
GGGGSGGGGSGGGGSGGGGSGGGGS
(Gly3Ser)₆ (SEQ ID NO: 82)
GGGSGGGSGGGSGGGSGGGSGGGS
(Gly₄Ser)₆ (SEQ ID NO: 83)
GGGGS GGGGGGGGSGGGGSGGGGSGGGGS
GSADDAKKDAAKKDAAKKDDAKKDDAGS (SEQ ID NO: 84)
GSADDAKKDAAKKDAAKKDDAKKDDAKKDAGS (SEQ ID NO: 85)
(Gly₃Gln)₂ (SEQ ID NO: 86)
GGGQGGGQ
(Gly₄Gln)₂ (SEQ ID NO: 87)
GGGGQGGGGQ
(Gly₃Gln)₃ (SEQ ID NO: 88)
GGGQGGGQGGGQ
(Gly₄Gln)₃ (SEQ ID NO: 89)
GGGGQGGGGQGGGGQ
(Gly₃Gln)₄ (SEQ ID NO: 90)
GGGQGGGQGGGQGGGQ
(Gly₄Gln)₄ (SEQ ID NO: 91)
GGGGQGGGGQGGGGQGGGGQ
(Gly₃Gln)₅ (SEQ ID NO: 92)
GGGQGGGQGGGQGGGQGGGQ
(Gly₄Gln)₅ (SEQ ID NO: 93)
GGGGQGGGGQGGGGQGGGGQGGGGQ
(Gly₃Gln)₆ (SEQ ID NO: 94)
GGGQGGGQGGGQGGGQGGGQGGGQ
(Gly₄Gln)₆ (SEQ ID NO: 95)
GGGGQGGGGQGGGGQGGGGQGGGGQGGGGQ
(Gly₃Ser)₂ (SEQ ID NO: 96)
GGGSGGGS
(Gly₄Ser)₂ (SEQ ID NO: 97)
GGGGSGGGGS
SGGGGSSGGGGS (SEQ ID NO: 98)

What is claimed:

1. A multispecific molecule comprising a first binding domain and a second binding domain, wherein the first binding domain binds immunoglobulin, and the second binding domain binds a recycling target.

2. The multispecific molecule of claim 1, wherein the first binding domain is an scFv, Fv, scFab, Fab′, or Fab and the second binding domain is an scFv, Fv, scFab, Fab′, or Fab.

3. The multispecific molecule of claim 1 or claim 2, wherein said first binding domain and/or second binding domain is an scFv.

4. The multispecific molecule of any one of claims 1-3, wherein the first binding domain and the second binding domain are each an scFv.

5. The multispecific molecule of claim 1 or claim 2, wherein said first binding domain and/or second binding domain is an scFab.

6. The multispecific molecule of any one of claims 1, 2, and 5, wherein the first binding domain and the second binding domain are each an scFab.

7. The multispecific molecule of claim 1 or claim 2, wherein said first binding domain and/or second binding domain is a Fab.

8. The multispecific molecule of any one of claims 1, 2, and 7, wherein the first binding domain and the second binding domain are each a Fab.

9. The multispecific molecule of claim 1 or claim 2, wherein said first binding domain is an scFv and the second binding domain is a Fab.

10. The multispecific molecule of claim 1 or claim 2, wherein said first binding domain is an scFv and the second binding domain is a scFab.

11. The multispecific molecule of claim 1 or claim 2, wherein said first binding domain is a Fab and the second binding domain is an scFv.

12. The multispecific molecule of claim 1 or claim 2, wherein said first binding domain is a scFab and the second binding domain is an scFv.

13. The multispecific molecule of claim 1 or claim 2, wherein said first binding domain is a Fab and the second binding domain is an scFab.

14. The multispecific molecule of claim 1 or claim 2, wherein said first binding domain is a scFab and the second binding domain is a Fab.

15. The multispecific molecule of any one of claims 1-14, wherein the first binding domain and second binding domain are connected via a linker.

16. The multispecific molecule of claim 15, wherein the linker is a polypeptide linker.

17. The multispecific molecule of claim 15, wherein the linker is a SG₄S linker.

18. The multispecific molecule of claim 15, wherein the linker comprises a sequence selected from the group consisting of (Gly₃Ser)₃ (SEQ ID NO: 76), (Gly₄Ser)₃ (SEQ ID NO: 77), (Gly₃Ser)₄ (SEQ ID NO: 78), (Gly₄Ser)₄ (SEQ ID NO: 79), (Gly₃Ser)₅ (SEQ ID NO: 80), (Gly₄Ser)₅ (SEQ ID NO: 81), (Gly₃Ser)₆ (SEQ ID NO: 82), (Gly₄Ser)₆ (SEQ ID NO: 83), GSADDAKKDAAKKDAAKKDDAKKDDAGS (SEQ ID NO: 84), GSADDAKKDAAKKDAAKKDDAKKDDAKKDAGS (SEQ ID NO: 6285 (Gly₃Gln)₂ (SEQ ID NO: 86), (Gly₄Gln)₂ (SEQ ID NO: 87), (Gly₃Gln)₃ (SEQ ID NO: 88), (Gly₄Gln)₃ (SEQ ID NO: 89), (Gly₃Gln)₄ (SEQ ID NO: 90), (Gly₄Gln)₄ (SEQ ID NO: 91), (Gly₃Gln)₅ (SEQ ID NO: 92), (Gly₄Gln)₅ (SEQ ID NO: 93), (Gly₃Gln)₆ (SEQ ID NO: 94), (Gly₄Gln)₆ (SEQ ID NO: 95), (Gly₃Ser)₂ (SEQ ID NO: 96), and (Gly₄Ser)₂ (SEQ ID NO: 97).

19. The multispecific molecule of any one of claims 1-18, wherein the immunoglobulin bound by the first domain is IgG, IgA, IgE, IgD, or IgM.

20. The multispecific molecule of claim 19, wherein the immunoglobulin is IgG or IgA.

21. The multispecific molecule of any one of claims 1-20, wherein the immunoglobulin is expressed on a plasma cell or on a B cell.

22. The multispecific molecule of any one of claims 1-20, wherein the immunoglobulin is circulating in blood.

23. The multispecific molecule of any one of claims 1-22, wherein the recycling target is ASGR1.

24. The multispecific molecule of any one of claims 1-23, wherein the multispecific molecule depletes at least 50%, 55%, 60%, 65%, 70%, 75%, 80%, 85%, 90%, 95%, 98%, 99%, or 100% of immunoglobulin in vivo.

25. The multispecific molecule of claim 24, wherein the multispecific molecule depletes at least 70% of immunoglobulin.

26. The multispecific molecule of any one of claims 1-25, wherein the multispecific molecule depletes immunoglobulin in a human, cynomolgus monkey, or mouse.

27. The multispecific molecule of any one of claims 24-26, wherein the immunoglobulin is depleted in less than 72 hours.

28. The multispecific molecule of any one of claims 1-27, wherein the multispecific molecule dissociates from the immunoglobulin in an endosome of a cell that expresses the recycling target.

29. The multispecific molecule of claim 28, wherein the multispecific molecule remains bound to the recycling target in the endosome and is recycled to the cell surface.

30. The multispecific molecule of any one of claims 1-27, wherein the multispecific molecule dissociates from the recycling target in an endosome of a cell that expresses the recycling target.

31. The multispecific molecule of any one of claims 1-30 for use in therapy.

32. The multispecific molecule of any one of claims 1-30 for use in treating autoantibody-induced disease.

33. The multispecific molecule of any one of claims 1-30 for the manufacture of a medicament for the treatment of autoantibody-induced disease.

34. The multispecific molecule of claim 32 or 33, wherein the autoantibody-induced disease is selected from the group consisting of myasthenia gravis, Guillain-Barre syndrome, epilepsy, autoimmune limbic encephalitis, spinal cord injury, pediatric autoimmune neuropsychiatric disorders associated with streptococcal infection, neuromyotonia, morvan syndrome, multiple sclerosis, pemphigus vulgaris, pemphigus foliaceus, bullous pemphigoid, epidermosysis bullosa acquisita, pemphigoig gestationis, mucous membrane pemphigoid, licen sclerosus, antiphospholipid syndrome, relapsing polychondritis, autoimmune anemia, idiopathic trombocytic purpura, autoimmune Grave's disease, dilated cardiomyopathy, vasculitis, goodpasture's syndrome, idiopathic membranous nephropathy, rheumatoid arthritis, and systemic lupus erythematosus.

35. A method of treating a patient having at least one autoantibody-induced disease, comprising administering to the patient an effective amount of the multispecific molecule of any one of claims 1-30.

36. The method of claim 35, wherein the autoantibody-induced disease is selected from the group consisting of myasthenia gravis, Guillain-Barre syndrome, epilepsy, autoimmune limbic encephalitis, spinal cord injury, pediatric autoimmune neuropsychiatric disorders associated with streptococcal infection, neuromyotonia, morvan syndrome, multiple sclerosis, pemphigus vulgaris, pemphigus foliaceus, bullous pemphigoid, epidermosysis bullosa acquisita, pemphigoig gestationis, mucous membrane pemphigoid, licen sclerosus, antiphospholipid syndrome, relapsing polychondritis, autoimmune anemia, idiopathic trombocytic purpura, autoimmune Grave's disease, dilated cardiomyopathy, vasculitis, goodpasture's syndrome, idiopathic membranous nephropathy, rheumatoid arthritis, and systemic lupus erythematosus.
